(19) United States
(12) Patent Application Publication
(10) Pub. No.: US 2003/0166138 A1
(43) Pub. Date: Sep. 4, 2003

(54) Cyclic peptides and analogs useful to treat allergies

(76) Inventors: Todd Kinsella, Fayetteville, NC (US); Cara Ohashi, San Francisco, CA (US); Dave Anderson, San Bruno, CA (US)

Correspondence Address: DORSEY & WHITNEY LLP, INTELLECTUAL PROPERTY DEPARTMENT, 4 EMBARCADERO CENTER, SUITE 3400, SAN FRANCISCO, CA, 94111, US

(21) Appl. No.: 10/197,927

(22) Filed: Jul. 16, 2002

Related U.S. Application Data
(60) Provisional application No. 60/358,827, filed on Feb. 21, 2002.

(51) Int. Cl. A61K 38/12; C07H 21/04; C07K 7/64; C12P 21/02; C12N 5/06

(52) U.S. Cl. 435/69.1; 514/9; 530/317; 435/320.1; 435/325; 536/23.5

(57) ABSTRACT

The present provides cyclic compounds capable of modulating IgE production, as well as IL-4 induced processes associated therewith, and methods of using the cyclic compounds in a variety of in vitro and in vitro contexts.

### 1. CROSS REFERENCE TO RELATED APPLICATIONS

This application claims benefit under 35 U.S.C. §119(e) to U.S. provisional application No. 60/358,827, filed Feb. 21, 2002, the contents of which are incorporated herein by reference.

### 2. FIELD OF THE INVENTION

The present invention relates to cyclic compounds that modulate IL-4 receptor-mediated IgE production, polynucleotides encoding peptide embodiments of such cyclic compounds and methods of using the cyclic compounds and polynucleotides in a variety of contexts, such as in the treatment or prevention of diseases associated with or caused or characterized by IgE production and/or accumulation.

### 3. BACKGROUND OF THE INVENTION

The immune system protects the body against invasion by foreign environmental agents such as microorganisms or their products, foods, chemicals, drugs, molds, pollen, animal hair or dander, etc. The ability of the immune system to protect the body against such foreign invaders may be innate or acquired.

The acquired immune response, which stems from exposure to the foreign invader, is extremely complex and involves numerous types of cells that interact with one another in myriad ways to express the full range of immune response. Two of these cell types come from a common lymphoid precursor cell but differentiate along different developmental lines. One line matures in the thymus (T-cells); the other line matures in the bone marrow (B-cells). Although T- and B-cells differ in many functional respects, they share one of the important properties of the immune response: they both exhibit specificity towards a foreign invader (antigen). Thus, the major recognition and reaction functions of the immune response are contained within the lymph cells.

A third cell type that participates in the acquired immune response is the class of cells referred to as antigen-presenting cells (APC). Unlike the T- and B-cells, the APC do not have antigen specificity. However, they play an important role in processing and presenting the antigen to the T-cells.

While the T- and B-cells are both involved in acquired immunity, they have different functions. Both T- and B-cells have antigen-specific receptors on their surfaces that, when bound by the antigen, activate the cells to release various products. In the case of B-cells, the surface receptors are immunoglobulins and the products released by the activated B-cells are immunoglobulins that have the same specificity for the antigen as the surface receptor immunoglobulins. In the case of activated T-cells, the products released are not the same as their surface receptor immunoglobulins, but are instead other molecules, called cytokines, that affect other cells and participate in the elimination of the antigen. One such cytokine, released by a class of T-cells called helper T-cells, is interleukin-4 (IL-4).

The immunoglobulins produced and released by B-cells must bind to a vast array of foreign invaders (antigens). All immunoglobulins share certain common structural features that enable them to: (1) recognize and bind specifically to a unique structural feature on an antigen (termed an epitope); and (2) perform a common biological function after binding the antigen. Basically, each immunoglobulin consists of two identical light (L) chains and two identical heavy (H) chains. The H chains are linked together via disulfide bridges. The portion of the immunoglobulin that binds the antigen includes the amino-terminal regions of both L and H chains. There are five major classes of H chains, termed α, δ, ε, γ and μ, providing five different isotypes of immunoglobulins: IgA, IgD, IgE, IgG and IgM. Although all five classes of immunoglobulins may possess precisely the same specificity for an antigen, they all have different biological functions.

While the immune system provides tremendous benefits in protecting the body against foreign invaders, particularly those that cause infectious diseases, its effects are sometimes damaging. For example, in the process of eliminating an invading foreign substance some tissue damage may occur, typically as a result of the accumulation of immunoglobulins with non-specific effects. Such damage is generally temporary, ceasing once the foreign invader has been eliminated. However, there are instances, such as in the case of hypersensitivity or allergic reactions, where the immune response directed against even innocuous agents such as inhaled pollen, inhaled mold spores, insect bite products, medications and even foods, is so powerful that it results in severe pathological consequences or symptoms.

Such hypersensitivity or allergic reactions are divided into four classes, designated types I-IV. The symptoms of the type I allergic reactions, called anaphylactic reactions or anaphylaxis, include the common symptoms associated with mild allergies, such as runny nose, watery eyes, etc., as well as the more dangerous, and often fatal, symptoms of difficulty in breathing (asthma), asphyxiation (typically due to constriction of smooth muscle around the bronchi in the lungs) and a sharp drop in blood pressure. Also included within the class of type I allergic reactions are atopic reactions, including atopic dermatitis, atopic eczema and atopic asthma.

Even when not lethal, such anaphylactic allergic reactions produce symptoms that interfere with the enjoyment of normal life. One need only witness the inability of an allergy sufferer to mow the lawn or hike through the woods to understand the disruptive force even mild allergies have on everyday life. Thus, while the immune system is quite beneficial, it would be desirable to be able to interrupt its response to invading foreign agents that pose no risk or threat to the body.

IgE immunoglobulins are crucial immune mediators of such anaphylactic hypersensitivity and allergic reactions, and have been shown to be responsible for the induction and maintenance of anaphylactic allergic symptoms. For example, anti-IgE antibodies have been shown to interfere with IgE function and alleviate allergic symptoms (Jardieu, 1995, Curr. Op. Immunol. 7:779-782; Shields et al., 1995, Int. Arch. Allergy Immunol. 107:308-312). Thus, release and/or accumulation of IgE immunoglobulins are believed to play a crucial role in the anaphylactic allergic response to innocuous foreign invaders. Other diseases associated with or mediated by IgE production and/or accumulation include, but are not limited to, allergic rhinitis, allergic conjunctivitis, systemic mastocytosis, hyper IgE syndrome, IgE gammopathies and B-cell lymphoma.

Although IgEs are produced and released by B-cells, the cells must be activated to do so (B-cells initially produce only IgD and IgM). Isotype switching of B-cells to produce IgE is a complex process that involves the replacement of certain immunoglobulin constant (C) regions with other C regions that have biologically distinct effector functions, without altering the specificity of the immunoglobulin. For IgE switching, a deletional rearrangement of the Ig H chain gene locus occurs, which results in the joining of the switch region of the μ gene, Sμ, with the corresponding region of the ε gene, Sε.

This IgE switching is induced in part by IL-4 (or IL-13) produced by T-cells. The IL-4 induction initiates transcription through the Sε region, resulting in the synthesis of germline (or “sterile”) ε transcripts (that is, transcripts of the unrearranged Cε H genes) that lead to the production of IgE, instead of IgM.

IL-4 induced germline ε transcription and consequent synthesis of IgE is inhibited by interferon gamma (IFN-γ), interferon alpha (IFN-α) and tumor growth factor beta (TGF-β). In addition to the IL-4 signal, a second signal, also normally delivered by T-cells, is required for switch recombination leading to the production of IgE. This second T-cell signal may be replaced by monoclonal antibodies to CD40, infection by Epstein-Barr virus or hydrocortisone.

Generally, traditional treatments for diseases mediated by IgE production and/or accumulation regulate the immune system following synthesis of IgE. For example, traditional therapies for the treatment of allergies include anti-IgE antibodies or anti-histamines designed to modulate the IgE-mediated response resulting in mast cell degranulation. Drugs are also known that generally downregulate IgE production or that inhibit switching of, but not induction of, germline ε transcription (see, e.g., Loh et al., 1996, J. Allerg. Clin. Immunol. 97(5):1141).

Although these treatments are often effective, treatments that act to reduce or eliminate IgE production altogether would be desirable. By reducing or eliminating IgE production, the hypersensitivity or allergic response may be reduced or eliminated altogether. Accordingly, the availability of compounds that are modulators of IgE production, such as compounds that are capable of modulating, and in particular inhibiting, IL-4 receptor mediated germline ε transcription and/or consequent IgE production, would be highly desirable.

### 4. SUMMARY OF THE INVENTION

These and other objects are furnished by the present invention, which in one aspect provides cyclic compounds which are capable of modulating, and in particular inhibiting, the IL-4 signaling cascade involved in the production of IgE. The cyclic compounds of the invention are generally 4 to 10 residue peptides or peptide analogs that are cyclized in a head-to-tail fashion such that the compounds do not have free termini.

The cyclic compounds of the invention inhibit IL-4 induced germline ε transcription in cellular assays. As a consequence of this activity, the cyclic compounds of the invention can be used to modulate the IL-4 signaling cascade involved in the production of IgE, and in particular to inhibit IL-4 induced germline ε transcription. Since these processes are involved in the production of IgE, the cyclic compounds of the invention can also be used to inhibit the production of IgE. In a specific embodiment, the cyclic compounds may be used to inhibit IL-4 induced IgE production as a therapeutic approach towards the treatment or prevention of diseases associated with or mediated by IgE production and/or accumulation, such as anaphylactic hypersensitivity or allergic reactions and/or symptoms associated with such reactions, allergic rhinitis, allergic conjunctivitis, systemic mastocytosis, hyper IgE syndrome, IgE gammapathies, B-cell lymphoma and atopic disorders such as atopic dermatitis, atopic eczema and/or atopic asthma.

In another aspect, the invention provides polynucleotides capable of expressing certain peptide embodiments of the cyclic compounds of the invention in cells. Such polynucleotides take advantage of the trans protein splicing reaction mediated by split inteins and generally comprise a first segment encoding a C-terminal intein domain, a second segment encoding a linear version of a cyclic peptide of the invention and a third segment encoding an N-terminal intein domain. The segments are arranged such that expression of the polynucleotide yields a precursor polypeptide that spontaneously excises and generates a cyclic peptide of the invention. The polynucleotide may further include, among other elements, a promoter upstream of the coding sequences which is operably linked to the coding sequences such that the coding sequences are under control of the promoter.

The polynucleotides may be incorporated into plasmids or expression vectors, including viral or retroviral vectors, which may then be introduced into suitable host cells, such as bacterial, yeast, insect and mammalian host cells. Such host cells may be used to express the cyclic peptide encoded by the polynucleotide. The polynucleotides, plasmids and/or expression vectors may also be introduced into packaging systems suitable for administration of the cyclic peptide encoded thereby to animals in the context of gene therapy.

Accordingly, in still another aspect, the invention provides host cells, or progeny thereof, comprising a polynucleotide, plasmid or expression vector of the invention. The cells may transiently express the polynucleotide, plasmid or expression vector or alternatively, the polynucleotide, plasmid or expression vector may be integrated into the genome of the host cell or progeny.

In yet another aspect, the invention provides pharmaceutical compositions comprising a cyclic compound or polynucleotide of the invention and a pharmaceutically acceptable carrier, excipient or diluent. Such compositions are particularly useful in the therapeutic methods of the invention.

In yet another aspect, the invention provides methods of modulating, and in particular inhibiting or downregulating, IgE production and/or processes mediated by or associated with IgE production and/or accumulation. Such processes include, but are not limited to, the IL-4 signaling cascade involved in isotype switching to produce IgE and IL-4 induced germline ε transcription. The method generally involves administering to a cell an amount of a cyclic compound of the invention effective to modulate IgE production and/or processes mediated by or associated therewith. The method may be practiced in vitro, in vivo or ex vivo. In one embodiment, the cyclic compound is administered to the cell by contacting the cell with a cyclic compound of the invention. In an alternative embodiment, certain peptide embodiments of the cyclic compounds are administered to the cell via a polynucleotide that expresses the cyclic peptide inside the cell.

In still another aspect, the invention provides methods of treating or preventing diseases associated with, or mediated or caused by, IgE production and/or accumulation. The method generally comprises administering to an animal suffering from such a disease an amount of a cyclic compound of the invention effective to treat or prevent the disease and/or one or more of its symptoms. In one embodiment, a cyclic compound of the invention is administered to the animal per se or in the form of a pharmaceutical composition of the invention. In an alternative embodiment, certain peptide embodiments of the cyclic compounds are administered to the animal utilizing gene therapy approaches, such as by the administration of polynucleotides or cells according to the invention. Diseases associated with, or mediated or caused by IgE production and/or accumulation that may be treated or prevented according to the methods of the invention include, but are not limited to, anaphylactic hypersensitivity or allergic reactions (including food and drug allergies) and/or symptoms associated therewith, allergic rhinitis, allergic conjunctivitis, systemic mastocytosis, hyper IgE syndrome, IgE gammopathies, B-cell lymphoma and atopic disorders such as atopic dermatitis, atopic eczema and/or atopic asthma. The method may be practiced therapeutically to treat the disease once the onset of the disease and/or its associated symptoms has already occurred, or prophylactically to prevent the onset of the disease and/or its associated symptoms. The methods may be practiced in veterinary contexts or in the treatment of humans.

### 5. BRIEF DESCRIPTION OF THE FIGURES.

FIG. 1 provides an illustration of an exemplary peptide embodiment of a cyclic compound of the invention demonstrating “head-to-tail” cyclization;

FIG. 2 provides a cartoon illustrating the protein splicing reaction mediated by a contiguous intein;

FIG. 3 provides a cartoon illustrating the presumed mechanism of action of the protein splicing reaction of FIG. 2;

FIG. 4 provides a cartoon illustrating the protein splicing reaction mediated by a split intein to generate a cyclic peptide;

FIG. 5 provides a cartoon illustrating the presumed mechanism of action of the protein splicing reaction of FIG. 4;

FIG. 6A provides a cartoon illustrating a “wild-type” polynucleotide intein construct capable of expressing a cyclic peptide;

FIG. 6B provides a cartoon illustrating a “mutant” intein construct incapable of expressing a cyclic peptide;

FIG. 7 provides the nucleotide sequence (coding strand) and translated amino acid sequence of the I_{C}-extein-I_{N}-BFP region of the retroviral vector of FIG. 6A (stop codons are indicated with “.”);

FIG. 8 provides a cartoon illustrating the diphtheria toxin selection and Tet or Dox-controlled expression features of the A5T4 reporter cell line;

FIG. 9 provides a cartoon outlining the selection and screening methodology used to identify certain 4- and 5-mer cyclic compounds of the invention;

FIG. 10 provides a cartoon outlining the two-round selection approach used for 5- and 6-mer peptide libraries;

FIG. 11 provides FACS profiles demonstrating the inhibitory activity of cyclic peptide cyclo(SRVEI) expressed using the wild-type intein of FIG. 6A and the lack of inhibitory activity of the mutant SRVEI construct expressed using the mutant intein construct of FIG. 6B in naive A5T4 cells;

FIG. 12 provides a graph comparing the GFP reporter ratios of certain peptides expressed using the wild-type intein construct of FIG. 6A (cyclic peptides) with those of the corresponding (non-cyclic) peptide expressed using the mutant intein construct of FIG. 6B;

FIG. 13 provides a graph comparing the relative fluorescence of endogenous epsilon promoter transcription of A5T4 clones expressing the wild-type intein construct of FIG. 6A in which Dox-regulatable expression has been turned on (−Dox/+IL-4) with clones in which Dox-regulatable expression has been turned off (+Dox/+IL-4);

FIG. 14 provides a graph comparing the relative fluorescence of endogenous epsilon promoter transcription of A5T4 clones expressing the mutant intein construct of FIG. 6B in which Dox-regulatable expression has been turned on (−Dox/+IL-4) with clones in which Dox-regulatable expression has been turned off (+Dox/+IL-4); and

FIG. 15, Panel A, provides MALDI-TOF mass spectrometry data (left side) and MS/MS data (right side) of cell lysates spiked with 100 pmol of synthetic cyclo(SRGDGWS); Panel B provides MALDI-TOF mass spectrometry data (left side) and MS/MS data (night side) of cell lysates of cells expressing the wild-type intein construct (and cyclic peptide) of Panel C.

### 6. DETAILED DESCRIPTION OF THE PREFERRED EMBODIMENTS

6.1 Abbreviations

The abbreviations used for the genetically encoded amino acids are conventional and are as follows:

| Amino Acid | Three-Letter | One-Letter |
|---|---|---|
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

When the three-letter abbreviations are used, unless specifically preceded by an “L” or a “D,” the amino acid may be in either the L- or D-configuration about α-carbon (C_{α}). For example, whereas “Ala” designates alanine without specifying the configuration about the α-carbon, “D-Ala” and “L-Ala” designate D-alanine and L-alanine, respectively. When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When peptide sequences are presented as a string of one-letter or three-letter abbreviations (or mixtures thereof), the sequences are presented in the N→C direction in accordance with common convention.

The abbreviations used for the genetically encoding nucleosides are conventional and are as follows: adenosine (A); guanosine (G); cytidine (C); thymidine (T); and uridine (U). Unless specifically delineated, the abbreviated nucleotides may be either ribonucleosides or 2′-deoxyribonucleosides. The nucleosides may be specified as being either ribonucleosides or 2′-deoxyribonucleosides on an individual basis or on an aggregate basis. When specified on an individual basis, the one-letter abbreviation is preceded by either a “d” or an “r,” where “d” indicates the nucleoside is a 2′-deoxyribonucleoside and “r” indicates the nucleoside is a ribonucleoside. For example, “dA” designates 2′-deoxyriboadenosine and “rA” designates riboadenosine. When specified on an aggregate basis, the particular nucleic acid or polynucleotide is identified as being either an RNA molecule or a DNA molecule. Nucleotides are abbreviated by adding a “p” to represent each phosphate, as well as whether the phosphates are attached to the 3′-position or the 5′-position of the sugar. Thus, 5′-nucleotides are abbreviated as “pN” and 3′-nucleotides are abbreviated as “Np,” where “N” represents A, G, C, T or U. When nucleic acid sequences are presented as a string of one-letter abbreviations, the sequences are presented in the 5′→3′ direction in accordance with common convention, and the phosphates are not indicated.

6.2 Definitions

As used throughout the instant application, the following terms shall have the following meanings:

“Polynucleotide” or “Nucleic Acid” refers to two or more nucleosides that are covalently linked together. The polynucleotide may be wholly comprised ribonucleosides (i.e., an RNA), wholly comprised of 2′-deoxyribonucleosides (i.e., a DNA) or mixtures of ribo- and 2′-deoxyribonucleosides. While the nucleosides will typically be linked together via standard phosphodiester linkages, the polynucleotides may include one or more nonstandard linkages. Non-limiting examples of such non-standard linkage include phosphoramidates (Beaucage et al., 1993, Tetrahedron 49:1925; Letsinger, 1970, J. Org. Chem. 35:3800; Sprinzl et al., 1977, Eur. J. Biochem. 81:579; Letsinger et al., 1986, Nucl. Acids Res. 14:3487; Sawai et al., 1984, Chem. Lett. N5:805-808; Letsinger et al., 1988, J. Am. Chem. Soc. 110:4470; Pauwels et al., 1986, Chemica Scripta 26:141), phosphorothioates (Mag et al., 1991, Nucl. Acids Res. 19:1437; U.S. Pat. No. 5,644,048), phosphorodithioates (Briu et al., 1989, J. Am. Chem. Soc. 111:2321), O-methylphosphodiesters (Eckstein, 1991, Oligonucleotides and Analogues: A Practical Approach, Oxford University Press), amides (Egholm, 1992, J. Am. Chem. Soc. 114:1895; Meier et al., 1992, Chem. Rut. Ed. Engl. 31:1008; Nielsen, 1993, Nature 365:366; Carlsson et al., 1996, Nature 380:207 WO 94/25477; WO 92/20702; U.S. Pat. Nos. 6,107,470; 5,786,461; 5,773,571; 5,719,262; and 5,539,082), positively-charged linkages (Denocy et al., 1995, Proc. Natl. Acad. Sci. USA 92:6097 and non-ionic linkages (U.S. Pat. Nos. 5,386,023; 5,637,684; 5,602,240; 5,216,141; and 4,469,863); Kiedrowski et al., 1991, Angew. Chem. Intl. Ed. English 30:423; Letsinger et al., 1988, J. Am. Chem. Soc. 110:4470; Letsinger et al., 1994, Nucleosides & Nucleotides 13:1597; Chapters 2 and 3, ASC Symposium Series 580, “Carbohydrate Modifications in Antisense Research,” Sanghui & Cook, Eds.; Mesmaeker et al., 1994, Bioorg. Med. Chem. Lett. 4:395; Jeffs et al., 1994, J. Biomolecular NMR 34:17; Mutti et al., 1996, Tetrahedron Lett. 37:8743-8746). Additional interlinkages are described in U.S. Pat. No. 6,033,909.

The polynucleotides may also include one or more interlinkages in which the ribose moieties of the nucleosides are replaced with other linkages, including the non-ribose polynucleotide interlinkages described in U.S. Pat. Nos. 5,235,033 and 5,034,506 and Chapters 6 and 7, ACS Symposium Series 580, supra, or which include carbocyclic sugars, such as those described in Jenkins et al., 1995, Chem Soc. Rev. pp. 169-176.

The polynucleotide may be single-stranded or double-stranded, or may include both single-stranded regions and double-stranded regions.

Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxenthine, etc. Preferably, such modified or synthetic nucleobases will be encoding nucleobases.

“Promoter” or “Promoter Sequence” refers to a polynucleotide regulatory region capable of initiating transcription of a downstream (3′ direction) coding sequence. A promoter typically includes a transcription initiation site (conveniently defined, for example, by mapping with nuclease S1) and protein binding domains responsible for binding proteins that initiate transcription.

“IL-4 Effector” refers to a molecule or compound that activates an IL-4 receptor signal transduction pathway. While not intending to be bound by any theory of operation, it is believed that such IL-4 effectors activate IL-4 receptor signal transduction by binding the IL-4 receptor, either alone or as part of a larger complex. Non-limiting examples of IL-4 effectors include IL-4 and IL-13.

“IL-4 Receptor-Mediated Transcription” refers to transcription effected as a consequence of an interaction between an IL-4 effector and the IL-4 receptor. Non-limiting examples of IL-4 receptor-mediated transcription include transcription of a germline ε promoter (defined below) induced by IL-4 and IL-13.

“IL-4 inducible promoter” refers to a promoter that initiates transcription when a cell comprising a nucleic acid molecule including such a promoter is exposed to, or contacted with, an IL-4 effector. While not intending to be bound by any particular theory of operation, it is believed that contacting a cell comprising such a promoter with an IL-4 effector causes the activation of a DNA-binding protein that then binds the IL-4 inducible promoter and induces transcription of coding sequences downstream of the promoter.

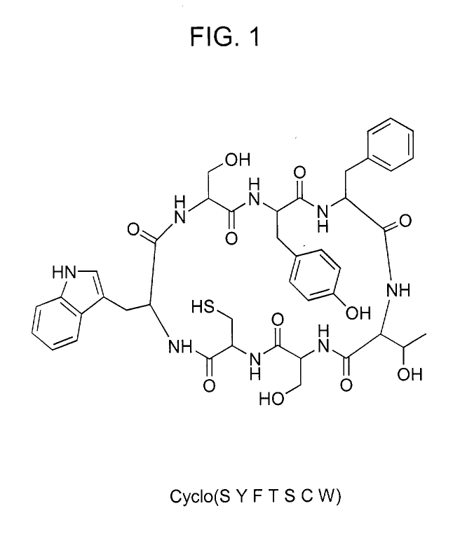

An “ε promoter” or a “germline ε promoter” is an IL-4 inducible promoter that, when induced in a B-cell, leads to the production of IgE immunoglobulins. Such IL-4 inducible germline ε promoters are well-known in the art, and include, by way of example and not limitation the engineered IL-4 inducible germline ε promoter described in FIG. 1A of WO 99/58663, incorporated herein by reference.

A compound that “modulates an IL-4 inducible germline ε promoter” or that “modulates IL-4 induced germline ε transcription” or that “modulates IL-4 receptor-mediated germline ε transcription” has the ability to change or alter the expression downstream of the germline ε promoter induced by an IL-4 effector such as IL-4 or IL-13. The change in downstream expression may occur at the mRNA (transcriptional) level or at the protein (translational) level. Hence, the change in downstream expression may be monitored at the RNA level, for example by quantifying induced downstream transcription products, or at the protein level, for example by quantifying induced downstream translation products. The compound may act to modulate the IL-4 inducible germline ε promoter via any mechanism of action. For example, the compound may act to modulate the IL-4 inducible germline 8 promoter by interacting with or binding a DNA binding protein involved in the IL-4 induced transcription, or by interacting with or binding the IL-4 inducible germline ε promoter per se.

“Intein” refers to a naturally occurring or artificially-created polypeptide or protein splicing element that mediates its excision from a precursor polypeptide or protein and the joining of the flanking polypeptide or protein sequences (“exteins”). A list of known inteins is published at http://www.neb.com/inteins.html; polynucleotides encoding such inteins are published at http://www.neb.com/inteins/int_reg.html. Unless specifically noted otherwise, an intein may be a “contiguous intein” which is composed of a single polypeptide chain or a “split intein” which is composed of two or more distinct polypeptide chains.

“Retro Peptide or Peptide Analog” refers to a peptide or peptide analog having a primary sequence that is the reverse (in the N→C direction) of the primary sequence of a corresponding parent peptide or peptide analog. For example, the retro peptide of a parent peptide having the primary sequence “SSLRW” is “WRLSS”.

“Inverso Peptide or Peptide Analog” refers to a peptide or peptide analog having a primary sequence that is identical to that of a corresponding parent peptide or peptide analog, but in which all chiral α-carbons are in the opposite configuration. For example, the inverso peptide of a parent peptide having the primary sequence “SSLRW” is “sslrw”.

“Retro-Inverso Peptide or Peptide Analog” refers to a peptide or peptide analog having the combined features of a retro peptide or peptide analog and an inverso peptide or peptide analog, as defined above. Retro-inverso peptides and peptide analogs may be achieved either by reversing the polarity of the peptide or analogous bonds of a corresponding parent peptide or peptide analog or by reversing the order of the primary sequence (in the NoC direction) and changing the chirality of the α-carbons of a corresponding parent peptide or peptide analog. For example, retro-inverso peptides of a parent peptide having the sequence “SSLRW” include the peptides “S˜S˜L˜R˜W”, where each “˜” represents a peptide bond having the polarity —C(O)—NH—, and wrlss.

“Alkyl” by itself or as part of another substituent refers to a saturated or unsaturated, branched, straight-chain or cyclic monovalent hydrocarbon group having the stated number of carbon atoms (i.e., C₁-C₆ means from one to six carbon atoms) derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. The expression “lower alkyl” refers to alkyl groups composed of from 1 to 6 carbon atoms.

“Alkanyl” by itself or as part of another substituent refers to a saturated branched, straight-chain or cyclic alkyl group. Typical alkanyl groups include, but are not limited to, methanyl; ethanyl; propanyls such as propan-1-yl, propan-2-yl (isopropyl), cyclopropan-1-yl, etc.; butyanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl” by itself or as part of another substituent refers to an unsaturated branched, straight-chain or cyclic alkyl group having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl group having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Heteroalkyl,” “Heteroalkanyl,” “Heteroalkenyl,” and “Heteroalkynyl by themselves or as part of another substituent refer to alkyl, alkanyl, alkenyl, and alkynyl groups, respectively, in which one or more of the carbon atoms are each independently replaced with the same or different heteratoms or heteroatomic groups. Typical heteratoms and/or heteroatomic groups which can be included in these groups include, but are not limited to, —O—, —S—, —O—O—, —S—S—, —S—, —NR—, ═N—N═, —N═N—, —N═N—NR—, —S(O)—, —S(O)₂—, —S(O)₂NR—, and the like, where each R is independently hydrogen or (C₁-C₆) alkyl.

“Parent Aromatic Ring System” refers to an unsaturated cyclic or polycyclic ring system having a conjugated n electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like

“Aryl” by itself or as part of another substituent refers to a monovalent aromatic hydrocarbon group having the stated number of carbon atoms (i.e., C5-C14 means from five to 14 carbon atoms) derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In preferred embodiments, the aryl group is (C₅-C₁₄) aryl, with (C₅-C₁₀) being even more preferred. Particularly preferred aryls are cyclopentadienyl, phenyl and naphthyl.

“Arylalkyl” by itself or as part of another substituent refers to an acyclic alkyl group in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylakenyl and/or arylalkynyl is used. In preferred embodiments, the arylalkyl group is (C₆-C₁₆) arylalkyl, e.g., the alkanyl, alkenyl or alkynyl moiety of the arylalkyl group is (C₁-C₆) and the aryl moiety is (C₅-C₁₀) In particularly preferred embodiments the arylalkyl group is (C₆-C₁₃), e.g., the alkanyl, alkenyl or alkynyl moiety of the arylalkyl group is (C₁-C₃) and the aryl moiety is (C₅-C₁₀)

“Parent Heteroaromatic Ring System” refers to a parent aromatic ring system in which one or more carbon atoms are each independently replaced with the same or different heteroatoms or heteroatomic groups. Typical heteratoms or heteroatomic groups to replace the carbon atoms include, but are not limited to, N, NH, P, O, S, Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Also included in the definition of “parent heteroaromatic ring system” are those recognized rings that include substituents, such as benzopyrone. Typical parent heteroaromatic ring systems include, but are not limited to, arsindole, benzodioxan, benzofuran, benzopyrone, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Heteroaryl” by itself or as part of another substituent refers to a monovalent heteroaromatic group having the stated number of ring atoms (i.e., “5 to 14 membered” means from 5 to 14 ring atoms) derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In preferred embodiments, the heteroaryl group is a 5-14 membered heteroaryl, with 5-10 membered heteroaryl being particularly preferred.

“Heteroarylalkyl” by itself or as part of another substituent refers to an acyclic alkyl group in which one of the hydrogen atoms bonded to a carbon atom, typically a terminal or Sp³ carbon atom, is replaced with a heteroaryl group. Where specific alkyl moieties are intended, the nomenclature heteroarylalkanyl, heteroarylakenyl and/or heterorylalkynyl is used. In preferred embodiments, the heteroarylalkyl group is a 6-20 membered heteroarylalkyl, e.g., the alkanyl, alkenyl or alkynyl moiety of the heteroarylalkyl is 1-6 membered and the heteroaryl moiety is a 5-14-membered heteroaryl. In particularly preferred embodiments, the heteroarylalkyl is a 6-13 membered heteroarylalkyl, e.g., the alkanyl, alkenyl or alkynyl moiety is 1-3 membered and the heteroaryl moiety is a 5-10 membered heteroaryl.

“Substituted Alkyl, Heteroalkyl, Aryl, Arylalkyl, Heteroaryl or Heteroarylalkyl” by themselves or as part of another substituent refers to an alkyl, heteroalkyl, aryl, arylalkyl, heteroaryl or heteroarylalkyl groups in which one or more hydrogen atoms is replaced with another substituent group. Exemplary substituent groups include, but are not limited to, —OR′, —SR′, —NR′R′, —NO₂, —NO, —CN, —CF₃, halogen (e.g., —F, —Cl, —Br and —I), —C(O)R′, —C(O)OR′, —C(O)NR′, —S(O)R′, —S(O)₂R′, —S(O)₂NR′R′, and the like, where each R′ is independently selected from the group consisting of hydrogen, (C₁-C₆) alkyl, (C₅-C 10) aryl, (C₆-C₁₆) arylalkyl, 5-10 membered heteroaryl and 6-16 membered heteroarylalkyl.

6.3 The Cyclic Compounds

The cyclic compounds of the invention are generally peptides and/or peptides analogs which, as will be discussed in more detail below, are capable of modulating a variety of processes involved in IL-4 receptor mediated isotype switching of B-cells to produce IgE. The cyclic compounds of the invention are composed of from 4 to 10 residues and are cyclized in a head-to-tail fashion such that the cyclic compounds do not have free termini. For example, a cyclic peptide is cyclized in a head-to-tail fashion when its amino-terminus is covalently bonded to its carboxy-terminus, either directly or by way of an optional linker, such that the resultant cyclic peptide has no free amino or carboxy terminus. An example of a cyclic peptide of the invention illustrating such head-to-tail cyclization is depicted in FIG. 1. For ease of reference, this head-to-tail cyclization is abbreviated using the notation “cyclo(Z),” where Z represents the sequence of the cyclized peptide or peptide analogue. As a specific example, the cyclic peptide of FIG. 1 is abbreviated as cyclo(SYFTSCW).

Thus, the cyclic compounds of the invention are generally 4 to 10 residue cyclic peptides or peptide analogs characterized by the formula (I):

cyclo(Xⁿ˜Xⁿ˜Xⁿ˜Xⁿ˜Xⁿ˜Xⁿ˜Xⁿ˜Xⁿ˜Xⁿ˜Xⁿ) (I)

wherein each “Xⁿ” independently represents an amino acid or residue bearing a side chain belonging to a certain designated class and each “˜” represents a linkage. The definitions of the various classes of amino acids or residues that define structure (I), and hence the cyclic compounds of the invention, are as follows:

“Hydrophilic Amino Acid or Residue” refers to an amino acid or residue having a side chain exhibiting a hydrophobicity of less than zero according to the normalized consensus hydrophobicity scale of Eisenberg et al., 1984, J. Mol. Biol. 179:125-142. Genetically encoded hydrophilic amino acids include L-Thr (T), L-Ser (S), L-His (H), L-Glu (E), L-Asn (N), L-Gln (O), L-Asp (D), L-Lys (K) and L-Arg (R).

“Acidic Amino Acid or Residue” refers to an amino acid or residue having a side chain exhibiting a pK value of less than about 6 when the amino acid is included in a peptide or polypeptide. Acidic amino acids typically have negatively charged side chains at physiological pH due to loss of a hydrogen ion. Genetically encoded acidic amino acids include L-Glu (E) and L-Asp (D).

“Basic Amino Acid or Residue” refers to an amino acid or residue having a side chain exhibiting a pK value of greater than about 6 when the amino acid is included in a peptide or polypeptide. Basic amino acids typically have positively charged side chains at physiological pH due to association with hydronium ion. Genetically encoded basic amino acids include L-His (H), L-Arg (R) and L-Lys (K).

“Polar Amino Acid or Residue” refers to an amino acid or residue having a side chain that is uncharged at physiological pH, but which has at least one bond in which the pair of electrons shared in common by two atoms is held more closely by one of the atoms. Genetically encoded polar amino acids include L-Asn (N), L-Gln (O), L-Ser (S) and L-Thr (T).

“Hydrophobic Amino Acid or Residue” refers to an amino acid or residue having a side chain exhibiting a hydrophobicity of greater than zero according to the normalized consensus hydrophobicity scale of Eisenberg et al., 1984, J. Mol. Biol. 179:125-142. Genetically encoded hydrophobic amino acids include L-Ile (I), L-Phe (F), L-Val (V), L-Leu (L), L-Trp (W), L-Met (M), L-Ala (A), Gly (G) and L-Tyr (Y).

“Aromatic Amino Acid or Residue” refers to an amino acid or residue having a side chain that includes at least one aryl or heteroaryl group. The aryl or heteroaryl group may include one or more of the same or different substituents such as —OH, —OR″, —SH, —SR″, —CN, halogen (e.g., —F, —Cl, —Br, —I), —NO₂, —NO, —NH₂, —NHR″, —NR″R″, —C(O)R″, —C(O)O⁻, —C(O)OH, —C(O)OR″, —C(O)NH₂, —C(O)NHR″, —C(O)NR″R″ and the like, where each R′ is independently (C₁-C₆) alkyl, substituted (C₁-C₆) alkyl, (C₂-C₆) alkenyl, substituted (C₂-C₆) alkenyl, (C₂-C₆) alkynyl, substituted (C₂-C₆) alkynyl, (C₅-C₁₀) aryl, substituted (C₅-C₁₀) aryl, (C₆-C₁₆) arylalkyl, substituted (C₆-C₁₆) arylakyl, 5-10 membered heteroaryl, substituted 5-10 membered heteroaryl, 6-16 membered heteroarylalkyl or substituted 6-16 membered heteroarylalkyl. Genetically encoded aromatic amino acids include L-His (H), L-Phe (F), L-Tyr (Y) and L-Trp (W).

“Non-polar Amino Acid or Residue” refers to an amino acid or residue having a side chain that is uncharged at physiological pH and which has bonds in which the pair of electrons shared in common by two atoms is generally held equally by each of the two atoms (i.e., the side chain is not polar). Genetically encoded non-polar amino acids include L-Leu (L), L-Val (V), L-Ile (I), L-Met (M), L-Gly (G) and L-Ala (A).

“Aliphatic Amino Acid or Residue” refers to an amino acid or residue having an aliphatic hydrocarbon side chain. Genetically encoded aliphatic amino acids include L-Ala (A), L-Val (V), L-Leu (L) and L-Ile (I).

“Hydroxyl-Containing Amino Acid or Residue” refers to an amino acid or residue which includes a non-aromatic hydroxyl group on its side chain. Genetically-encoded hydroxyl-containing amino acids include L-Ser (S) and L-Thr (T).

“Small Amino Acid or Residue” refers to an amino acid or residue having a side chain that is composed of a total three or fewer carbon and/or heteroatoms (excluding sulfur). The small amino acids or residues may be further categorized as aliphatic, non-polar, polar, acidic or hydroxyl-containing small amino acids or residues, in accordance with the above definitions. Genetically-encoded small amino acids include Gly (G), L-Ala (A), L-Val (V), L-Ser (S) and L-Thr (T).

The amino acid L-Cys (C) is unusual in that it can form disulfide bridges with other L-Cys (C) amino acids or other sulfanyl-containing amino acids (referred to as “cysteine-like” amino acids or residues). The ability of L-Cys (C) (and other cysteine-like amino acids or residues) to exist in a peptide or peptide analog in either the reduced free —SH or oxidized disulfide-bridged form affects whether L-Cys (C) contributes net hydrophobic or hydrophilic character to the peptide or analog. While L-Cys (C) exhibits a hydrophobicity of 0.29 according to the normalized consensus scale of Eisenberg (Eisenberg et al., 1984, supra), it is to be understood that for purposes of the present invention L-Cys (C) is categorized as a polar hydrophilic amino acid, notwithstanding the general classifications defined above.

The amino acid Gly (G) is unusual in that it bears no side chain on its α-carbon and, as a consequence, contributes only a peptide linkage to the particular peptide of which it is a part. Moreover, owing to the lack of a side chain, it is the only genetically-encoded amino acid having an achiral α-carbon. For purposes of the present invention, although it does not have a side chain, Gly may be included as an aliphatic amino acid or residue.

Although it exhibits a hydrophobicity of 0.12 according to the normalized consensus scale of Eisenberg et al. (Eisenberg et al., 1984, supra), owing to its cyclic structure, the amino acid proline defines a class of amino acids or residues referred to as “structurally constrained” amino acids or residues. Such structurally constrained amino acids or residues typically include a substituent at the amino nitrogen or, like proline, include the amino nitrogen in a ring structure.

As will be appreciated by those of skill in the art, the above-defined categories are not mutually exclusive. Indeed, the delineated category of small amino acids includes amino acids from all of the other delineated categories except the aromatic category. Thus, amino acids having side chains exhibiting two or more physico-chemical properties can be included in multiple categories. As a specific example, amino acid side chains having heteroaromatic moieties that include ionizable heteroatoms, such as His, may exhibit both aromatic properties and basic properties, and can therefore be included in both the aromatic and basic categories. The appropriate classification of any amino acid or residue will be apparent to those of skill in the art, especially in light of the detailed disclosure provided herein.

While the above-defined categories have been exemplified in terms of the genetically encoded amino acids, the cyclic compounds of the invention are not restricted to the genetically encoded amino acids. In addition to the genetically encoded amino acids, the cyclic compounds of the invention may be comprised, either in whole or in part, of naturally-occurring and/or synthetic non-encoded amino acids. Certain commonly encountered non-encoded amino acids of which the cyclic compounds of the invention may be comprised include, but are not limited to: the D-enantiomers of the genetically-encoded amino acids; 2,3-diaminopropionic acid (Dpr); α-aminoisobutyric acid (Aib); ε-aminohexanoic acid (Aha); δ-aminovaleric acid (Ava); N-methylglycine or sarcosine (MeGly or Sar); ornithine (Orn); citrulline (Cit); t-butylalanine (Bua); t-butylglycine (Bug); N-methylisoleucine (MeIle); phenylglycine (Phg); cyclohexylalanine (Cha); norleucine (Nle); naphthylalanine (NaI); 2-chlorophenylalanine (Ocf); 3-chlorophenylalanine (Mcf); 4-chlorophenylalanine (Pct); 2-fluorophenylalanine (Off); 3-fluorophenylalanine (Mff); 4-fluorophenylalanine (Pff); 2-bromophenylalanine (Obf); 3-bromophenylalanine (Mbf); 4-bromophenylalanine (Pbf); 2-methylphenylalanine (Omf); 3-methylphenylalanine (Mmf); 4-methylphenylalanine (Pmf); 2-nitrophenylalanine (Onf); 3-nitrophenylalanine (Mnf); 4-nitrophenylalanine (Pnf); 2-cyanophenylalanine (Ocf); 3-cyanophenylalanine (Mcf); 4-cyanophenylalanine (Pcf); 2-trifluoromethylphenylalanine (Otf); 3-trifluoromethylphenylalanine (Mtf); 4-trifluoromethylphenylalanine (Ptf); 4-aminophenylalanine (Paf); 4-ioclophenylalanine (Pif); 4-aminomethylphenylalanine (Pamf); 2,4-dichlorophenylalamine (Opef); 3,4-dichlorophenylalanine (Mpcf); 2,4-difluorophenylalanine (Opff); 3,4-difluorophenylalanine (Mpff); pyrid-2-ylalanine (2pAla); pyrid-3-ylalanine (3pAla); pyrid-4-ylalanine (4pAla); naphth-1-ylalanine (1nAla); naphth-2-ylalanine (2nAla); thiazolylalanine (taAla); benzothienylalanine (bAla); thienylalanine (tAla); furylalanine (fAla); homophenylalanine (hPhe); homotyrosine (hTyr); homotryptophan (hTrp); pentafluorophenylalanine (5ff); styrylkalanine (sAla); authrylalanine (aAla); 3,3-diphenylalanine (Dfa); 3-amino-5-phenypentanoic acid (Afp); penicillamine (Pen); 1,2,3,4-tetrahydroisoquinoline-3-carboxylic acid (Tic); β-2-thienylalanine (Thi); methionine sulfoxide (Mso); N(w)-nitroarginine (nArg); homolysine (hLys); phosphonomethylphenylalanine (pmPhe); phosphoserine (pSer); phosphothreonine (pThr); homoaspartic acid (hAsp); homoglutanic acid (hGlu); 1-aminocyclopent-(2 or 3)-ene-4 carboxylic acid; pipecolic acid (PA), azetidine-3-carboxylic acid (ACA); 1-aminocyclopentane-3-carboxylic acid; allylglycine (aOly); propargylglycine (pgGly); homoalanine (hAla); norvaline (nVal); homoleucine (hLeu), homovaline (hVal); homoisolencine (hIle); homoarginine (hArg); N-acetyl lysine (AcLys); 2,4-diaminobutyric acid (Dbu); 2,3-diaminobutyric acid (Dab); N-methylvaline (MeVal); homocysteine (hCys); homoserine (hSer); hydroxyproline (Hyp) and homoproline (hPro). Additional non-encoded amino acids of which the cyclic compounds of the invention may be comprised will be apparent to those of skill in the art (see, e.g., the various amino acids provided in Fasman, 1989, CRC Practical Handbook of Biochemistry and Molecular Biology, CRC Press, Boca Raton, Fla., at pp. 3-70 and the references cited therein, all of which are incorporated by reference). These amino acids may be in either the L- or D-configuration.

Those of skill in the art will recognize that amino acids bearing side chain protecting groups may also comprise the cyclic compounds of the invention. Non-limiting examples of such protected amino acids, which in this case belong to the aromatic category, include (protecting groups listed in parentheses): Arg(tos), Cys(methylbenzyl), Cys (nitropyridinesulfenyl), Glu(6-benzylester), Gln(xanthyl), Asn(N-6-xanthyl), His(bom), His(benzyl), His(tos), Lys(fmoc), Lys(tos), Ser(O-benzyl), Thr (O-benzyl) and Tyr(O-benzyl).

Non-limiting examples of non-encoding structurally-constrained amino acids of which the cyclic compounds of the invention may be composed include, but are not limited to, D-Pro; N-methyl amino acids (L-configuration); 1-aminocyclopent-(2 or 3)-ene-4-carboxylic acid; pipecolic acid; azetidine-3-carboxylic acid; homeproline (hPro); and 1-aminocyclopentane-3-carboxylic acid.

Other amino acids not specifically mentioned herein can be readily categorized based on their observed physical and chemical properties in light of the definitions provided herein.

In the cyclic compounds of structure (I), the symbol “˜” between each specified residue Xⁿ designates a backbone constitutive linking moiety. When the cyclic compounds of the invention are peptides, each “˜” between the various Xⁿ represents an amide or peptide linkage having the following polarity: —C(O)—NH—. It is to be understood, however, that the cyclic compounds of the invention include analogs of peptides in which one or more amide or peptide linkages are replaced with a linkage other than an amide or peptide linkage, such as a substituted amide linkage, an isostere of an amide linkage, or a peptide or amide mimetic linkage. Thus, when used in connection with defining the various Xⁿ comprising the cyclic compounds of the invention, the term “residue” refers to the C_{α} carbon and side chain moiety(ies) of the designated amino acid or class of amino acid. As a specific example, defining an Xₙ as being a “Gly residue” means that Xₙ is C_{α}H₂. Defining Xₙ as being an “Ala residue” means that Xₙ is C_{α}HCH₃ in which the C_{α} carbon is in either the D- or L-configuration. Defining an Xⁿ as being an “A residue” means that Xⁿ is C_{α}HCH₃ in which the C_{α} carbon is in the L-configuration.

Substituted amide linkages that may be included in the cyclic compounds of the invention include, but are not limited to, groups of the formula —C(O)—NR²—, where R² is (C₁-C₆) alkyl, (C₅-C₁₀) aryl, substituted (C₅-C₁₀) aryl, (C₆-C₁₆) arylalkyl, substituted (C₆-C₁₆) arylalkyl, 5-10 membered heteroaryl, substituted 5-10 membered heteroaryl, 6-16 membered heteroarylalkyl or substituted 6-16 membered heteroarylalkyl. In a specific embodiment, R² is (C₁-C₆) alkanyl, (C₂-C₆) alkenyl, (C₂-C₆) alkynyl or phenyl.

Isosteres of amides that may be included in the cyclic compounds of the invention include, but are not limited to, hydroxymethyl carbonyl, —NR³—SO—, —NR³—S(O)₂—, —CH₂—CH₂—, —CH═CH— (cis and trans), —CH₂—NH—, —CH₂—S—, —CH₂—O—, —C(O)—CH₂—, —CH(OH)—CH₂—, —CH(OH)—C(O)—, —CH(OH)—CH(OH)— and —CH₂—S(O)₂, where R³ is hydrogen or R², where R² is as previously defined. These interlinkages may be included in the cyclic compounds of the invention in either the depicted polarity or in the reverse polarity. An additional isostere of an amide of which the cyclic compounds may be composed include a reversed-polarity amide or substituted amide of the formula —NR³—C(O)—, where R³ is as previously defined. Peptide analogs including such non-amide linkages, as well as methods of synthesizing such analogs, are well-known. See, for example, Spatola, 1983, “Peptide Backbone Modifications,” In: Chemistry and Biochemistry of Amino Acids, Peptides and Proteins, Weinstein, Ed., Marcel Dekker, New York (general review); Morley, 1980, Trends Pharm. Sci. 1:463-468; Hudson et al., 1979, Int. J. Prot. Res. 14:177-185 (—CH₂—NH—, —CH₂—CH₂); Spatola et al., 1986, Life Sci. 38:1243-1249 (CCH₂—S—); Hann, 1982, J. Chem. Soc. Parkin Trans. I. 1:307-314 (—CH═CH—, cis and trans); Almquist et al., 1980, J. Med. Chem. 23:1392-1398 (—C(O)—CH₂—); Jennings-White et al., 1982, Tetrahedron Lett. 23:2533-2534 (C(O)—CH₂—); European Patent Application EP 45665; Chemical Abstracts CA 97:39405 (CH(OH)CH₂—); Holladay et al., 1983, Tetrahedron Lett. 24:4401-4404 (—CH(OH)—CH₂—); and Hruby, 1982, Life Sci. 31:189-199 (—CH₂—S—); Kiso, 1996, Biopolymers 40:235-244 (hydroxymethyl carbonyl); Morse et al., 1995, Int. J. Pept. Protein Res. 45:501-507 (—S(O)₂—NH—); Thanki et al., 1992 Protein Sci. 1(8): 1061-1072 (—CH(OH)—CH(OH)—). The —S(O)—NH— isostere can be prepared by reduction of —S(O)₂—NH—.

Alternatively, one or more amide linkages may be replaced with peptidomimetic and/or amide mimetic moieties. Such mimetics include, for example, the linkages described in Olson et al., 1993, J. Med. Chem. 36:3039-3049; Ripka & Rich, 1998, Curr. Opin. Chem. Biol. 2:441-452; Borchardt et al., 1997, Adv. Drug. Deliv. Rev. 27:235-256 and the various references cited therein.

One or more linkages “˜”, but typically one linkage “˜” in the cyclic compounds of structure (I) may also represent a linker. Such linkers may be useful in situations where the cyclic compounds, owing in part to their small size, are under significant conformational strain when composed wholly of amide linkages or the other linkages described above. Such linkers may be composed of virtually any combination of atoms suitable for linking two ends of a peptide or peptide analog together. Preferred linkers include flexible moieties such as saturated hydrocarbons and ethers or polyethers. In a specific embodiment, one linkage “i” in structure (I) represents —C(O)(CH₂)ₘ—NH—, —C(O)(CH₂)ₘ—O—, —C(O)—(CH₂)ₘ—S, —C(O)—(CH₂—O—CH₂)ₚ—NH—, —C(O)—(CH₂—O—CH₂)ₚ—O— or —C(O)—(CH₂—O—CH₂)ₚ—S—, where m is an integer from 3 to 5 and p is an integer from 1 to 3. Other suitable linkers, as well as methods of synthesizing cyclic compounds including such linkers, will be apparent to those of skill in the art.

While structure (T) contains 10 specified residue positions, it is to be understood that the cyclic compounds of the invention may contain fewer than 10 residues. Indeed, many cyclic compounds of the invention are composed of 4, 5, 6 or 7 residues. Moreover, the various Xⁿ residues comprising the cyclic compounds of the invention may be in either the L- or D-configuration about their C_{α} carbons. In one embodiment, all of the chiral C_{α} carbons of a particular cyclic compound are in the same configuration. Additional specific embodiments of the cyclic compounds of the invention are described below.

In one embodiment, the cyclic compounds are 4 to 10 residue cyclic peptides or cyclic peptide analogs comprising a contiguous sequence of residues selected from the group consisting of structures (Ia)-(Ie):

X²⁶˜X²⁵˜X²¹˜X²⁵; (Ia)

X²²˜X²⁴˜X²⁷˜X²⁹; (Ib)

X²¹˜X²²˜X²⁴˜X²⁹; (Ic)

X²²˜X²¹˜X²¹˜X²⁹; and (Id)

X²⁸˜Glu˜X²¹˜X²⁹ (Ie)

wherein:

each X²¹ is independently a small residue;

each X²² is independently a basic residue;

each X²⁴ is independently a hydrophobic residue;

each X²⁵ is independently an aromatic residue;

each X²⁶ is independently a polar residue;

each X²⁷ is independently a small or a cysteine-like residue;

each X²⁸ is independently a small or an acidic residue; and

each X²⁹ is independently any residue.

In another embodiment, the cyclic compounds are 4 to 7-residue cyclic peptides or cyclic peptide analogs selected from structures (IIa)-(IIe):

cyclo(X²⁶˜X²⁵˜X²¹˜X²⁵˜X³⁰˜X³⁰˜X³⁰) (IIa)

cyclo(X²²˜X²⁴˜X²⁷˜X²⁹˜X³⁰˜X³⁰˜X³⁰) (IIb)

cyclo(X²¹˜X²²˜X²⁴˜X²⁹˜X³⁰˜X³⁰˜X³⁰) (IIc)

cyclo(X²²˜X²¹˜X²¹˜X²⁹˜X³⁰˜X³⁰˜X³⁰) (IId)

cyclo(X²⁸˜Glu˜X²¹˜X²⁹˜X³⁰˜X³⁰˜X³⁰) (IIe)

wherein:

X²¹, X²², X²³, X²⁴, X²⁵, X²⁶, X²⁷, X²⁸ and X²⁹ are as previously defined for structures (Ia-Ie) and each X³⁰ is independently present or absent, and if present, is any residue.

In another embodiment, the cyclic compounds are cyclic peptides or cyclic peptide analogs according to structure (Ia) or (IIa) in which:

each X²¹ is independently selected from the group consisting of a Ser, a Thr, a Cys and an Asn residue;

each X²⁵ is independently selected from the group consisting of a Trp, a Phe, a Tyr and a His residue; and/or

each X²⁶ is independently selected from the group consisting of a Ser, a Thr, a Cys and a Asn residue.

In still another embodiment, the cyclic compounds are cyclic peptide analogs according to structure (IIa) which have the structure (IIa′):

cyclo(X⁴⁰˜X⁴¹˜X⁴²˜X⁴³˜X³⁰˜X³⁰˜X³⁰) (IIa′)

wherein:

X³⁰ is as previously defined;

X⁴⁰ is a Ser, a Thr, a Cys or a Asn residue (preferably Thr, Cys or Asn);

X⁴¹ is a Trp, a Phe, a Tyr or a His residue (preferably Trp or Phe);

X⁴² is a Ser or a Thr residue; and/or

X⁴³ is a Phe, a Tyr, a His or a Trp residue (preferably Phe, Tyr or His).

In one embodiment of the compounds of structure (Ia), (IIa) or (IIa′); the compound is composed of 5, 6 or 7 residues.

In another embodiment of the compounds of structure (IIa) or (IIa′), X³⁰˜X³⁰˜X³⁰ is selected from the group consisting of aromatic˜small˜small, small˜basic, aliphatic, Phe˜Thr˜Ser, Ser˜Arg, Ser and Val.

In another embodiment, the compounds of structure (IIa′) are selected from the group consisting of cyclo(TWSFV) (cyclo(SEQ ID NO:1)), cyclo(CWSYFTS) (cyclo(SEQ ID NO:5)), cyclo(NWSHSR) (cyclo(SEQ ID NO:9)), cyclo(NFTFS) (cyclo(SEQ ID NO:10)) and retro and retro-inverso peptides and peptide analogs thereof.

In still another embodiment, the cyclic compounds are cyclic peptides or peptide analogs according to structure (Ib) or (IIb) composed of 5, 6 or 7 residues.

In still another embodiment, the cyclic compounds are cyclic peptides or cyclic peptide analogs according to structure (Ib) or (IIb) in which:

X²² is an Arg, a Lys or an Orn residue (preferably Arg);

X²⁴ is an aromatic residue (preferably Trp, Phe, Tyr or His) or an aliphatic residue (preferably Ile or Leu); and/or

X²⁷ is a small hydroxyl-containing residue or a Cys residue.

In one embodiment of the compounds of structure (IIb), X²⁹˜X³⁰˜X³⁰˜X³⁰ is selected from the group consisting of hydroxyl-containing˜aliphatic (preferably Ser˜Leu), aliphatic˜Asn (preferably Leu˜Asn), basic˜basic˜acidic (preferably Arg˜Arg˜Asp) and Cys˜basic aromatic˜small hydroxyl-containing˜basic (preferably Cys˜His˜Ser˜Arg).

In yet another embodiment, the compounds of structure (IIb) are selected from the group consisting of cyclo(RWSSL) (cyclo(SEQ ID NO:6)), cyclo(RISLN) (cyclo(SEQ ID No:4)), cyclo(RISRRD) (cyclo(SEQ ID NO:15)) and retro and retro-inverso peptides and peptide analogs thereof.

In still another embodiment, the cyclic compounds are cyclic peptides or cyclic peptide analogs according to structure (Ic) or (IIc) that are composed of 5 residues.

In yet another embodiment of the compounds of structure (IIc), X²⁹˜X³⁰˜X³⁰˜X³⁰ is selected from the group consisting of acidic˜aliphatic (preferably Glu˜Ile) and aliphatic˜hydroxyl-containing (preferably Val˜Ser) and the compound has one or more features selected from the group consisting of:

X²¹ is a small hydroxyl-containing residue (preferably Ser or Thr) or a small aliphatic residue (preferably Ala);

X²² is an Arg, a Lys or an Orn residue; and

X²⁴ is a small aliphatic residue (preferably Val) or a hydrophobic residue (preferably Phe).

In yet another embodiment, the compounds of structure (IIc) are selected from the group consisting of cyclo(SRVEI) (cyclo(SEQ ID NO:2)), cyclo(ARFVS) (cyclo(SEQ ID NO:3)) and retro and retro-inverso peptides and peptide analogs thereof.

In yet another embodiment, the cyclic compounds are cyclic peptides or cyclic peptide analogs according to structure (Id) or (IId) that are composed of 5 residues. In another embodiment of the compounds of structure (IId) X^{29˜X}³⁰˜X³⁰˜X³⁰ is selected from the group consisting of aromatic˜small (preferably Phe˜Gly), small˜structurally constrained (preferably Gly˜Pro), Thr˜Met, Cys˜Met and Ser˜Met and has one or more characteristics selected from the group consisting of:

X²² is an Arg residue; and

each X²¹ is independently a small hydroxyl-containing residue (preferably Ser or Thr).

In still another embodiment, the cyclic compounds according to structure (IId) are selected from the group consisting of cyclo(RSSFG) (cyclo(SEQ ID NO:7)), cyclo(RSTGP) (cyclo(SEQ ID NO:16)) and retro-inverso and reversed peptides thereof.

In yet another embodiment, the cyclic compounds are cyclic peptides or cyclic peptide analogs according to structure (Ie) or (IIe) having the structure (IIe′):

cyclo (X⁵⁰˜Glu˜X⁵¹˜X²⁹˜X³⁰˜X³⁰˜X³⁰) (IIe′)

wherein:

X²⁹ and X³⁰ are as previously defined;

X⁵⁰ is a Glu, a Ser or an Ala residue; and/or

X⁵¹ is a Ser or an Ala residue.

In still another embodiment, the cyclic compound is cyclic peptide or cyclic peptide analog according to structure (Ie), (IIe) or (IIe′) which is composed of 4 or 5 residues. In yet another embodiment, the compounds are cyclic peptides or cyclic peptide analogs according to structure (IIe) or (IIe′) in which X²⁹˜X³⁰˜X³⁰˜X³⁰ is selected from the group consisting of aromatic (preferably Tyr), aliphatic˜aliphatic (preferably Val˜Ile), small hydroxyl-containing˜aromatic (preferably Ser˜Trp) and acidic˜aromatic (preferably Asp˜His).

In yet another embodiment, the compounds according to structure (IIe) or (IIe′), are selected from the group consisting of cyclo(EQSVI) (cyclo(SEQ ID NO:13)), cyclo(SQSY) (cyclo(SEQ ID NO:14)), cyclo(AQASW) (cyclo(SEQ ID NO:19)), cyclo(SQSDH) (cyclo(SEQ ID NO:20)) and retro and retro-inverso peptides and peptide analogs thereof.

In still another embodiment, the cyclic compounds of the invention exclude one or more of the following compounds: cyclo(SRGDGWS) (cyclo(SEQ ID NO:54)), cyclo(SRGPGWS) (cyclo(SEQ ID NO:55)), cyclo(SGRGDGWGS) (cyclo(SEQ ID NO:56)), cyclo(SSCMR) (cyclo(SEQ ID NO:21)), cyclo(SSFT) (cyclo(SEQ ID NO:22)), cyclo(SRRHCCH) (cyclo(SEQ ID NO:23)), and/or the various analogs and/or retro- and/or retro-inverso forms thereof.

In yet another embodiment, the cyclic compounds of the invention are cyclic peptides or cyclic peptide analogs selected from the group consisting of Compounds 1-20:

cyclo(X¹⁶˜X⁵˜X¹⁸˜X¹⁷˜X¹⁹); 1

cyclo(X¹⁶˜X¹⁵˜X¹⁸˜X⁴˜X⁸); 2

cyclo(X¹⁶˜X¹˜X¹⁵˜X⁵˜X¹⁸); 3

cyclo(X¹⁶˜X¹⁰˜X¹²˜X¹⁵˜X⁸); 4

cyclo(X¹⁶˜X²⁰˜X⁵˜X¹⁷˜X¹⁶˜X²˜X¹⁹); 5

cyclo(X¹⁶˜X¹⁶˜X¹⁰˜X¹⁵˜X¹⁹); 6

cyclo(X¹⁶˜X⁵˜X⁶˜X¹⁵˜X¹⁶); 7

cyclo(X¹⁶˜X⁴˜X¹¹˜X⁵˜X¹⁶˜X⁸˜X¹⁹); 8

cyclo(X¹⁶˜X⁴˜X¹¹˜X⁵˜X¹⁶˜X⁷); 9

cyclo(X¹⁶˜X¹²˜X⁵˜X¹⁷˜X⁵); 10

cyclo(X¹⁶˜X¹²˜X⁸˜X¹³˜X¹⁴); 11

cyclo(X¹⁶˜X⁶˜X¹˜X³˜X¹⁶); 12

cyclo(X¹⁶˜X¹⁸˜X⁸˜X⁴˜X¹⁴); 13

cyclo(X¹⁶˜X²⁰˜X¹⁶˜X¹⁴); 14

cyclo(X¹⁶˜X¹⁵˜X¹⁵˜X³˜X¹⁵˜X⁸); 15

cyclo(X¹⁶˜X¹⁷˜X⁶˜X¹³ ˜X¹⁵); 16

cyclo(X¹⁶˜X¹⁸˜X¹⁸˜X¹⁷˜X¹⁵); 17

cyclo(X¹⁶˜X¹³˜X¹⁹˜X⁹˜X¹⁰˜X¹⁸˜X⁶); ˜

cyclo(X¹⁶˜X¹⁶˜X¹˜X¹⁴˜X¹); 19

cyclo(X¹⁶˜X³˜X⁷˜X¹⁶˜X¹⁴); 20

wherein

X¹ is an Ala residue;

X² is a Cys residue;

X³ is an Asp residue;

X⁴ is a Glu residue;

X⁵ is a Phe residue;

X⁶ is a Gly residue;

X⁷ is a His residue;

X⁸ is an Ile residue;

X⁹ is a Lys residue;

X¹⁰ is a Leu residue;

X¹¹ is a Met residue;

X¹² is an Asn residue;

X¹³ is a Pro residue;

X¹⁴ is a Glu residue;

X¹⁵ is an Arg residue;

X¹⁶ is a Ser residue;

X¹⁷ is a Thr residue;

X¹⁸ is a Val residue;

X¹⁹ is a Trp residue; and

X²⁰ is a Tyr residue.

In yet another embodiment, the cyclic compounds are retro or retro-inverso peptides or peptide analogs of compounds 1-20.

In still another embodiment, the cyclic compounds are conservative mutants of compounds 1-20 in which 1, 2 or 3 residues are conservatively substituted with another residue of the same class, or a retro or retro-inverso peptide or peptide analog of such a conservative mutant.

In still another embodiment, the cyclic compounds of the invention are any of the previously-described embodiments which are cyclic peptides. In one embodiment, the cyclic peptide is composed wholly of gene-encoded L-amino acids. In another embodiment, the cyclic peptide is a retro or retro-inverso peptide of the previous embodiment.

In a final embodiment, the cyclic compounds are cyclic peptides selected from the group consisting of:

| | |
|---|---|
| cyclo(S F V T W); | cyclo(SEQ ID NO:1) |
| cyclo(S R V E I); | cyclo(SEQ ID NO:2) |
| cyclo(S A R F V); | cyclo(SEQ ID NO:3) |
| cyclo(S L N R I); | cyclo(SEQ ID NO:4) |
| cyclo(S Y F T S C W); | cyclo(SEQ ID NO:5) |
| cyclo(S S L R W); | cyclo(SEQ ID NO:6) |
| cyclo(S F G R S); | cyclo(SEQ ID NO:7) |
| cyclo(S E M F S I Q); | cyclo(SEQ ID NO:8) |
| cyclo(S R N W S H); | cyclo(SEQ ID NO:9) |
| cyclo(S N F T F); | cyclo(SEQ ID NO:10) |
| cyclo(S N I P Q); | cyclo(SEQ ID NO:11) |
| cyclo(S G A D S); | cyclo(SEQ ID NO:12) |
| cyclo(S V I E Q); | cyclo(SEQ ID NO:13) |
| cyclo(S Y S Q); | cyclo(SEQ ID NO:14) |
| cyclo(S R R D R I); | cyclo(SEQ ID NO:15) |
| cyclo(S T G P R); | cyclo(SEQ ID NO:16) |
| cyclo(S V V T R); | cyclo(SEQ ID NO:17) |
| cyclo(S P W K L V G); | cyclo(SEQ ID NO:18) |
| cyclo(S W A Q A); | cyclo(SEQ ID NO:19) |
| cyclo(S D H S Q); | cyclo(SEQ ID NO:20) |

and retro and retro-inverso peptides thereof.

Although the cyclic compounds of the invention are expected to be generally stable in vivo when used therapeutically, the in vivo stability of cyclic peptide embodiments of the cyclic compounds may be improved by including non-native peptide linkages at positions susceptible to cleavage by proteases or other degradative enzymes or agents. In one embodiment, the in vivo stability of cyclic peptide embodiments of the cyclic compounds towards tryptic-like proteases may be improved by replacing the native peptide bond before each Lys or Arg residue with a non-peptide bond, such as an isostere of an amide, a substituted amide or a peptidomimetic linkage. In a specific embodiment, these native peptide bonds are replaced with peptide bonds having a reversed polarity.

In another embodiment, the in vivo stability of cyclic peptide embodiments of the cyclic compounds towards chymotryptic-like proteases may be improved by replacing the native peptide bond before aromatic or aliphatic residues with a non-peptide bond, such as an isostere of an amide, a substituted amide or a peptidomimetic linkage. In a specific embodiment, these native peptide bonds are replaced with a peptide bond having a reversed polarity.

In still another embodiment, the in vivo stability of cyclic peptide embodiments of the cyclic compounds towards elastases may be improved, thereby improving the oral bioavailability of the cyclic peptides, by replacing the peptide bond before each small or medium-sized aliphatic residue or non-polar residue with a non-native peptide bond, such as an isostere of an amide, a substituted amide or a peptidomimetic linkage. In a specific embodiment, these native peptide bonds are replaced with a peptide bond having a reversed polarity.

In yet another embodiment, the cyclic compounds are cyclic peptide analogs in which each peptide bond has a reversed polarity. In a specific embodiment, such cyclic peptide analogs are retro-inverso peptide analogs of any of the previously-described embodiments of the cyclic compounds that are composed wholly of L-amino acids (i.e., all-L retro-inverso peptide analogs).

The cyclic compounds of the invention may be modified or derivatized in a variety of different ways to impart the compounds with specified properties. For example, the cyclic compounds may be modified or derivatized to include labels so as to enhance the utility of the cyclic compounds in, inter alia, screening assays. The label may be a direct label, i.e., a label that itself is detectable or produces a detectable signal, or it may be an indirect label, i.e., a label that is detectable or produces a detectable signal in the presence of another compound. The method of detection will depend upon the label used, and will be apparent to those of skill in the art.

Examples of suitable direct labels include radiolables, fluorophores, chromophores, chelating agents, particles, chemiluminescent agents and the like. Suitable radiolabels include, by way of example and not limitation, ³H, ¹⁴C, ³²P, ³⁵S, ³⁶Cl, ⁵⁷Co, ⁵⁸Co, ⁵⁹ Fe, ⁹⁰Y, ¹²⁵I, ¹³¹I and ¹⁸⁶Re. Suitable fluorophores include, by way of example and not limitation, fluorescein, rhodamine, phycoerythrin, Texas red, free or chelated lanthamide series salts such as Eu³⁺ and the myriad fluorophores available from Molecular Probes Inc., Eugene, Oreg. Examples of suitable colored labels include, by way of example and not limitation, metallic sol particles, for example, gold sol particles such as those described by Leuvering (U.S. Pat. No. 4,313,734); dye sole particles such as described by Gribnau et al. (U.S. Pat. No. 4,373,932) and May et al. (WO 88/08534); dyed latex such as those described Snyder (EP 0 280 559 and 0 281 327) and dyes encapsulated in liposomes as described by Campbell et al. (U.S. Pat. No. 4,703,017). Other direct labels that may be used will be apparent to those of skill in the art.

Examples of suitable indirect labels include enzymes capable of reacting with or interacting with a substrate to produce a detectable signal (such as those used in ELISA and EMIT immunoassays), ligands capable of binding a labeled moiety (for example biotin which binds a labeled streptavidin or avidin), and the like. Suitable enzymes useful as indirect labels include, by way of example and not limitation, alkaline phosphatase, horseradish peroxidase, lysozyme, glucose-6-phosphate dehydrogenase, lactate dehydrogenase and urease. The use of these enzymes in ELISA and EMIT immunoassays is described in detail in Engvall, 1980, Methods in Enzymology 70:419-439 and U.S. Pat. No. 4,857,453.

Methods and chemistries suitable for labeling the cyclic compounds of the invention are well-known. In one embodiment, the label is attached to a side chain bearing a functional group capable of reacting with a functional group on the label. Suitable functional groups on the cyclic compound side chains include, but are not limited to, amino, hydroxyl, sulfanyl, carboxyl, esters and the like. For example, a cyclic compound comprising a Lys residue may be labeled with a flurophose such as fluorescein by incubating the cyclic compound with, for example, fluroescein isothiocyanate, using conventional techniques. Alternatively, cyclic compounds composed wholly of gene-encoded amino acids may be labeled metabolically by culturing cells that express the cyclic compound in the presence of culture medium supplemented with a metabolic label, such as, by way of example and not limitation, [³⁵S]-methionine, one or more [¹⁴C]-labeled amino acids, one or more [¹⁵N]-labeled amino acids and/or one or more [³H]-labeled amino acids (with the tritium substituted at non-labile positions). Methods for carrying out such metabolic labeling are well-known in the art.

In another embodiment, the cyclic compounds may be derivatized to include affinity tags useful for, among other things, affinity extraction of the cyclic compound from, among other things, screening experiments (for example screening experiments designed to identify binding partners). Such affinity tags may include, by way of example and not limitation, biotin and other specific ligands, epitopes, histidine tags, and the like. Such tags may be attached to side chains, for example side chains of Cys, Lys, Asp and/or Gln residues, using standard techniques. Specific examples of biotin tags that may be used to derivatize the cyclic compounds of the invention are commercially available from Molecular Probes (Eugene Oreg.).

The cyclic compounds of the invention may also be derivatized to include agents or moieties that enhance the ability of the cyclic compounds to traverse cell membranes. Suitable agents and/or moieties are well-known in the art and may be attached to side chain functional groups of the cyclic compounds using standard techniques. In a specific embodiment, the cyclic compound is derivatized to include a signal peptide capable of effecting transport across a membrane. When combined with the stability-enhancing features described above, such derivatized cyclic compounds are particularly useful for in vivo therapeutic uses, as such compounds should exhibit not only good blood, serum and in vivo stability, but should also readily traverse cell membranes. The signal peptide may be attached to a side chain functional group of the cyclic compound via its N- or C-terminus, or alternatively, by way of a side chain functional group, using standard techniques. For example, the signal peptide may be attached by way of its carboxyl terminus to a side chain amino group of a Lys residue of the cyclic compound. Signal peptides capable of transporting compounds across cell membranes are well-known in the art. Any of these signal peptides may be used to derivatize the cyclic compounds of the invention. Specific examples of signal peptides which may be used include, by way of example and not limitation, HIV Tat sequences (see, e.g., Fawell et al, 1994, Proc. Natl. Acad. Sci. USA 91:664; Frankel et al., 1988, Cell 55:1189; Savion et al., 1981, J. Biol. Chem. 256:1149; Derossi et al., 1994, J. Biol. Chem. 269:10444; Baldin et al., 1990, EMBO J. 9:1511; U.S. Pat. No. 5,804,604; U.S. Pat. No. 5,670,617; and U.S. Pat. No. 5,652,122, the disclosures of which are incorporated herein by reference), antennapedia sequences (see, e.g., Garcia-Echeverria et al., 2001, Bioorg. Med. Chem. Lett. 11:1363-1366; Prochiantz, 1999, Ann. NY Acad. Sci. 886:172-179; Prochiantz, 1996, Curr. Opin. Neurobiol. 6:629-634; U.S. Pat. No. 6,080,724, and the references cited in all of the above, the disclosures of which are incorporated herein by reference) and poly(Arg) or poly(Lys) chains of 5-10 residues. Additional non-limiting examples of specific sequences can be found in U.S. Pat. No. 6,248,558; U.S. Pat. No. 6,043,339; U.S. Pat. No. 5,807,746 U.S. Pat. No. 6,251,398; U.S. Pat. No. 6,184,038 and U.S. Pat. No. 6,017,735, the disclosures of which are incorporated herein by reference.

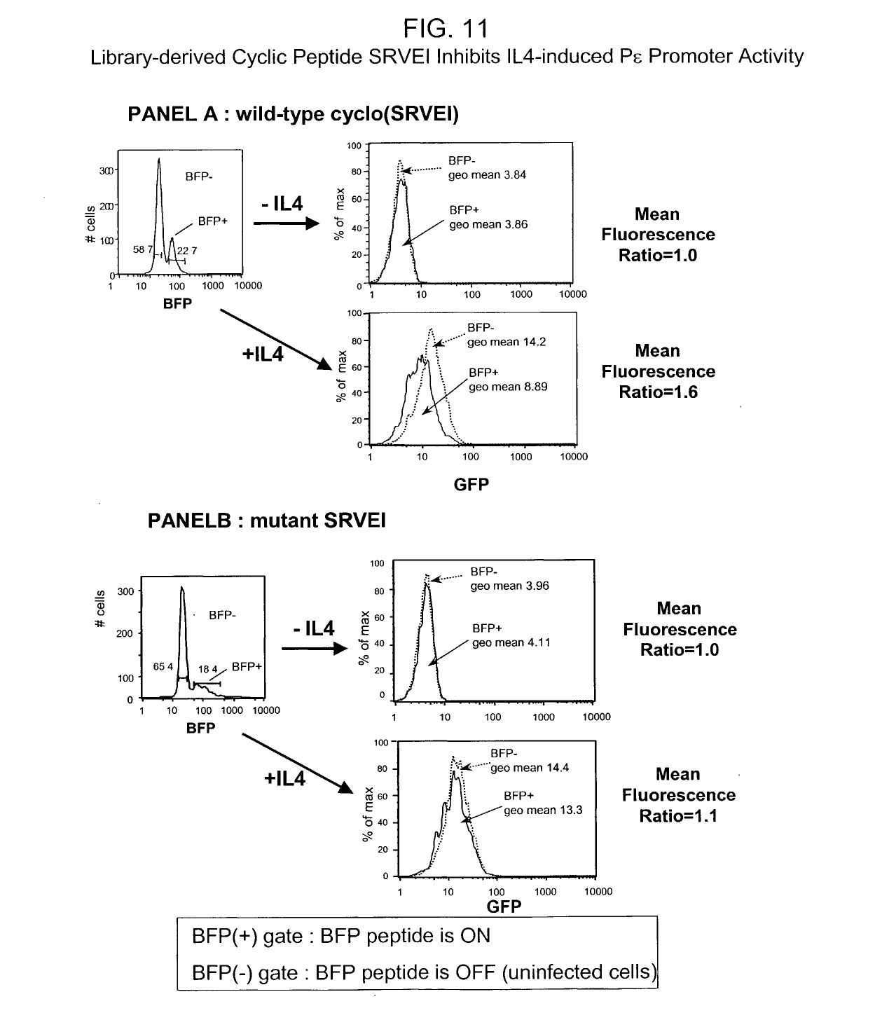

Active cyclic compounds of the invention are those that inhibit or downregulate IL-4 receptor-mediated or IL-4 induced IgE production and/or accumulation and/or processes associated therewith. The cyclic compounds of the invention may be assessed for such activity in any standard assay that measures the ability of a compound to modulate Il-4 induced IgE production and/or accumulation. For example, a cyclic compound of the invention may be administered to a human or animal B-cell (e.g., primary B-cells from blood, tonsils, spleens and other lymphoid tissues) stimulated with IL-4 (available from Pharmingen, Hamburg, Germany) and anti-CD40 nmAbs (available from Ancell Corporation, Bayport Minn.), and the amount of IgE produced measured, for example, by an ELISA technique, such as the ELISA technique described in Worm et al., 1998, Blood 92:1713. For cyclic compounds that readily traverse cell membranes, the compound may be administered to the cell by contacting the cell with the compound. Cyclic peptides composed wholly of gene encoded amino acids that do not readily traverse cell membranes may be administered to the cell using polynucleotides capable of expressing the cyclic peptide in conjunction with well-known delivery techniques (such polynucleotides are described in more detail, below). In one embodiment, such cyclic peptides may be administered using the well-known retroviral vectors and infection techniques pioneered by Richard Mulligan and David Baltimore with Psi-2 lines and analogous retroviral packaging systems based upon NIH 3T3 cells (see Mann et al., 1993, Cell 33:153-159, the disclosure of which is incorporated herein by reference). Such helper-defective packaging cell lines are capable of producing all of the necessary trans proteins (gag, pol and env) required for packaging, processing, reverse transcribing and integrating genomes. Those RNA molecules that have in cis the ψ packaging signal are packaged into maturing retrovirions. Virtually any of the art-known retroviral vectors and/or transfection systems may be used. Specific non-limiting examples of suitable transfection systems include those described in WO 97/27213; WO 97/27212; Choate et al., 1996, Human Gene Therapy 7:2247-2253; Kinsella et al., 1996, Human Gene Therapy 7:1405-1413; Hofmann et al., 1996, Proc. Natl. Acac. Sci. USA 93:5185-5190; Kitamura et al., 1995, Proc. Natl. Acac. Sci. USA 92:9146-9150; WO 94/19478; Pear et al., 1993, Proc. Natl. Acac. Sci. USA 90:8392-8396; Mann et al., 1993, Cell 33:153-159 and the references cited in all of the above, the disclosures of which are incorporated herein by reference. Specific non-limiting examples of suitable retroviral vector systems include vectors based upon murine stem cell virus (MSCV) as described in Hawley et al., 1994, Gene Therapy 1:136; vectors based upon a modified MGF virus as described in Rivere et al., 1995, Genetics 92:6733; pBABE as described in WO 97/27213 and WO 97/27212; and the vectors depicted in FIG. 11 of WO 01/34806, the disclosures of which are incorporated herein by reference. Other suitable vectors and/or transfection techniques are discussed in connection with gene therapy administration, infra. 101341 A specific assay for assessing IgE production that may be used to assay cyclic compounds of the invention is described in Worm et al., 2001, Int. Arch. Allergy Immunol. 124:233-236. Generally, a cyclic compound inhibits IgE production if it yields a decrease in measured IgE levels of about at least about 25% as compared to control cells (i.e., cells activated with anti-CD40 antibodies and IL-4 but not exposed to the cyclic compound). Skilled artisans will appreciate that cyclic compounds that inhibit greater levels of IL-4 induced IgE production, for example on the order of 50%, 60%, 70%, 80%, 90%, or even more as compared to control cells, are particularly desirable. Thus, while cyclic compounds that inhibit at lease about 25% of IL-4 induced IgE production as compared to control cells are active, compounds that inhibit at least about 50%, 75% or even more IL-4 induced IgE production as compared to control cells are preferred.

In another embodiment, cyclic compounds may be assayed for the ability to inhibit IL-4 induced transcription of a germline ε promoter. Generally, such assays involve administering a cyclic compound to an IL-4 induced cell comprising an IL-4 inducible germline ε promoter and assessing the amount of gene expression downstream of the ε promoter. Depending upon the ability of the cyclic compound to traverse cell membranes, it may be administered to the cell by contacting the cell with the cyclic compound or via the retroviral or other transfraction techniques described supra. The amount of the downstream gene expression may be assessed at the mRNA level, for example by quantifying the amount of a downstream transcription product produced, or at the translation level, for example by quantifying the amount of a downstream translation product produced. In one embodiment, the germline ε promoter is operably linked to a reporter gene that encodes a protein that produces an observable and/or detectable signal, such as a fluorescent protein. Specific examples of suitable assays for assessing cyclic compounds are described in U.S. Pat. No. 5,958,707, WO 01/66565, WO 01/34806, WO99/58663 and the Examples section.

Generally, a cyclic compound inhibits germline ε transcription if it yields a decrease in measured downstream expression of at least about 25% as compared to control cells activated with IL-4 but not exposed to the cyclic compound. Skilled artisans will appreciate that cyclic compounds that inhibit greater levels of IL-4 induced germline ε transcription, for example on the order of 50%, 60%, 70%, 80%, 90%, or even more as compared to control cells, are particularly desirable. Thus, while cyclic compounds that inhibit at least about 25% of IL-4 induced germline 6 transcription as compared to control cells are active, cyclic compounds that inhibit at least about 50%, 75% or even more IL-4 induced germline ε transcription as compared to control cells are preferred. In one embodiment of the invention, active cyclic compounds will have a reporter ratio of at least about >1.1 using the assays described in the Examples section. Particularly useful cyclic compounds are those having reporter ratios of ≧1.12, ≧1.13, ≧1.14, ≧1.15 or even higher.

As mentioned previously, B-cells initially produce IgD and IgM immunoglobulins and, when induced by the proper cytokines, produce IgEs. B-cells can be induced to produce other types of immunoglobulins, such as IgGs and IgAs, as well. For example, in the presence of the cytokine interleukin-2 (IL-2), B-cells produce IgG1; in the presence of a combination of IL-2 and TGF-β, B-cells produce IgA. In many situations, it is desirable to selectively inhibit the production of a single immunoglobulin isotype, as such specificity permits the ability to treat or prevent diseases associated with the production and/or accumulation of the specified immunoglobulin isotype without suppressing the immune system generally. Thus, in one embodiment, the cyclic compounds specifically inhibit IL-4 induced germline ε transcription or IL-4 induced IgE production and/or accumulation. By “specific” is meant that the cyclic compound inhibits IL-4 induced IgE production and/or accumulation or IL-4 induced germline ε transcription but does not significantly inhibit the production and/or accumulation of another immunoglobulin, or transcription of the promoter of another Ig isotype. A cyclic compound does not significantly inhibit production and/or accumulation of another Ig isotype, or transcription of another Ig isotype promoter, if the observed inhibition in an appropriate assay is on the order of 10% or less as compared to control cells. Such specificity may be with respect to a single Ig isotype, or may be with respect to one or more Ig isotypes. For example, a cyclic compound may be assessed for specificity by assaying its ability to inhibit, for example, IgA production and/or accumulation or to inhibit germline α transcription in assays similar to those described above, except that the cells are activated with effectors suitable for IgA switching and synthesis and the amount of IgA produced or the amount of expression downstream of a germline a promoter is assessed. A specific assay for assessing the ability of cyclic compounds to inhibit IgA production and/or TGF-β induced transcription of a germline a promoter is provided in the Examples section. Specific, non-limiting examples of cyclic compounds that specifically inhibit IL-4 induced IgE production and/or IL-4 induced germline ε transcription include the cyclic peptides cyclo(SEQ ID NO:1) through cyclo(SEQ ID NO:20).

6.4 Polynucleotides

The invention also includes polynucleotides capable of generating or expressing certain cyclic peptide embodiments of the cyclic compounds of the invention in vitro and/or in vivo. Such polynucleotides generate or express the cyclic peptides utilizing the trans splicing ability of split inteins.

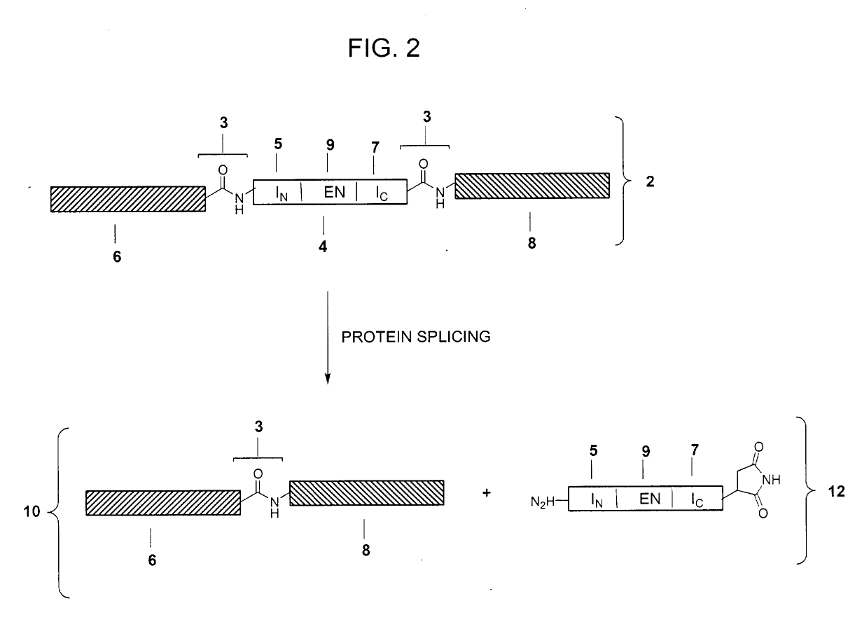

Inteins are protein splicing elements that mediate their excision from precursor proteins and the joining of the flanking proteins (N-extein and C-extein) to yield two mature proteins: the intein and the ligated protein (Perler et al., 1994, Nucl. Acids Res. 22:1125-1127). The bond formed between the ligated exteins is a native peptide bond (Perler et al., 1997, Curr. Opin. Chem. Biol. 1:292-299). The self-catalytic splicing reaction requires four nucleophilic displacements mediated by three conserved splice junction residues: (i) a Ser, Cys or Ala at the intein N-terminus; (ii) an Asn or Asp at the intein C-terminus; and (iii) a Ser, Thr or Cys at the beginning of the C-extein (Xu et al., 1993, Cell 75:1371-1377; Xu et al., 1994, EMBO J. 13:5517-5522; Shao et al. 1995, Biochemistry 34:10844-10850; Chong et al., 1996, J. Biol. Chem. 271:22159-22168; Xu & Perler, 1996, EMBO J. 15:5146-5153). An example of an intein-mediated protein splicing reaction is illustrated in FIG. 2. Referring to FIG. 2, precursor protein 2, which comprises an intein 4 flanked by an N-terminal extein 6 and a C-terminal extein 8, undergoes a protein splicing reaction, which is mediated or catalyzed by intein 4, to yield the excised intein 12 and fusion protein 10. As illustrated, intein 4 comprises an N-terminal protein splicing domain 5 (I_{N}) and a C-terminal protein splicing domain 7 (I_{C}) flanking an endonuclease domain 9 (EN). Fusion protein 10 comprises N-terminal extein 6 fused to C-terminal extein 8 via a native peptide bond 3.

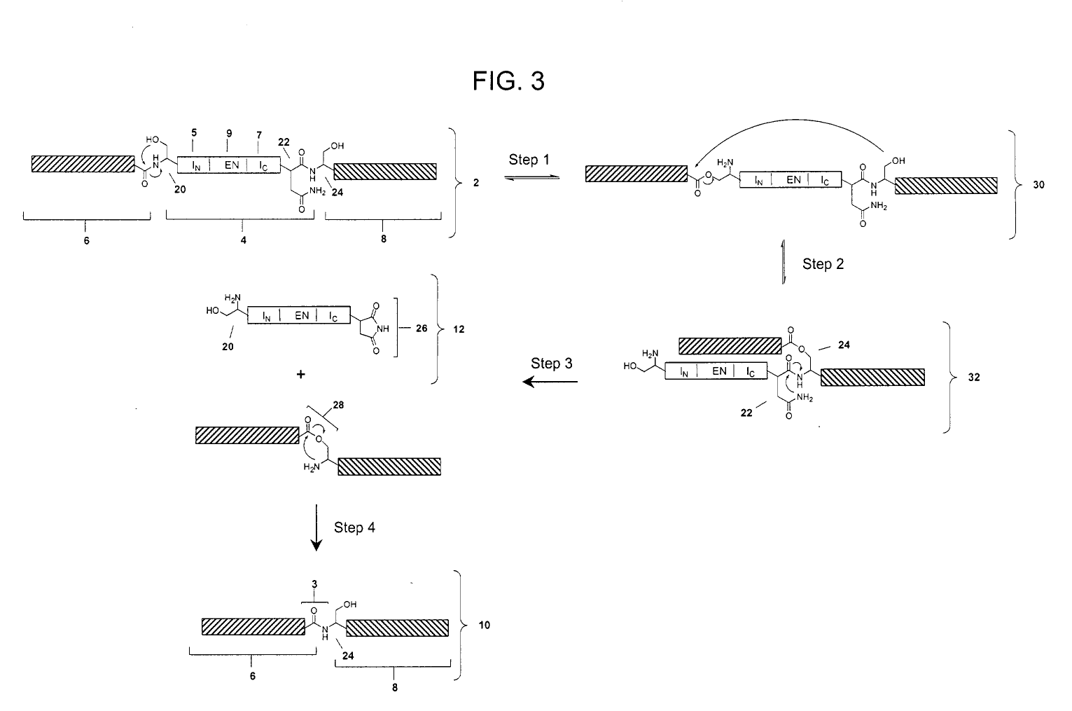

The mechanism of action of the intein-mediated protein splicing reaction is illustrated in FIG. 3. Referring to FIG. 3, which illustrates the precursor protein 2 of FIG. 2 showing a required Ser 20 at the intein N-terminus, the required Asn 22 at the intein C-terminus, and a required Ser 24 at the N-terminus (beginning) of the C-terminal extein 8, the splicing reaction proceeds via four steps: (1) Step 1 is an N—O acyl shift that occurs between the C-terminal amino acid of N-terminal extein 6 and the N-terminal Ser 20 of intein 4, resulting in the formation of a reactive ester 30; (2) in Step 2, the ester 30 is the focus of a transesterfication reaction that results in a branched intermediate 32 in which N-terminal extein 6 is attached via an ester to the N-terminal Ser 24 of C-terminal extein 8; (iii) in Step 3, branched intermediate 32 is resolved by the cyclization of the intein C-terminal Asn residue 22 to form a succinimide group 26, the excised intein 12 is released from precursor 2 and N-terminal extein 6 and C-terminal extein 8 are joined by an ester bond 28; (iv) in Step 4, a spontaneous O—N acyl shift generates a native peptide bond 3 between exteins 6 and 8 at the condensation point to yield fusion protein 10.

Split inteins, whether naturally occurring or artificially created by splitting a contiguous intein into two distinct “halves,” are capable of catalyzing a trans ligation reaction that yields an extein product cyclized in a head-to-tail fashion (see, e.g., Southworth et al., 1998, EMBO J. 17:918-926; Xu et al., 1999, Proc. Natl. Acad. Sci. USA 95:6705-6710; Evans et al., 1999, Biochemistry 274:18359-18363; Scott et al., 1999, Proc. Natl. Acad. Sci. USA 96:13638-13643). The splitting of an intein to catalyze a trans ligation reaction generally requires segregating the Iand I_{C} protein splicing domains and reversing their translational order such that the splicing and ligation reaction fuses the former N- and C-termini. This process is well-understood and is described, for example, in WO 01/66565 (see, e.g., FIGS. 1A, 1B, 2A and 2B and the text associated therewith).

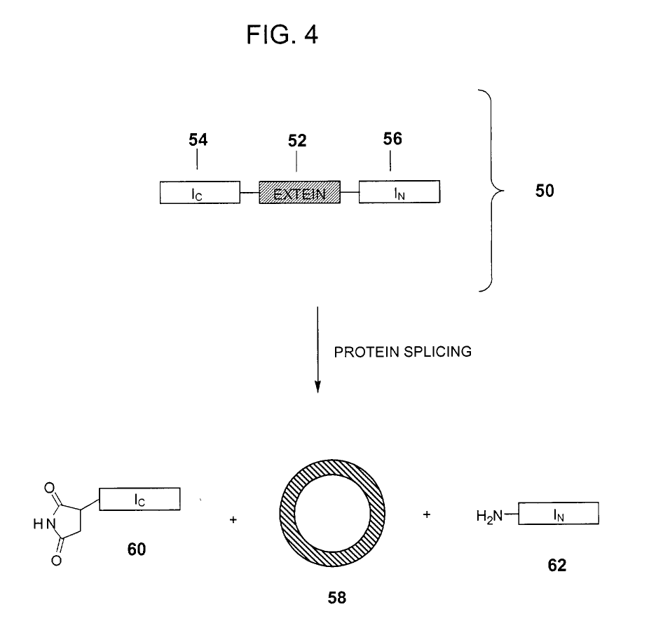

An example of such a split intein construct and the cyclization reaction catalyzed thereby is illustrated in FIG. 4. Referring to FIG. 4, precursor protein 50 comprises an extein 52 interposed between two intein protein splicing domains 54, 56. The upstream intein domain 54 corresponds to the C-terminal (I_{C}) domain 7 of FIG. 2. The downstream intein domain 56 corresponds to the N-terminal (I_{N}) domain 5 of FIG. 2.

The intein illustrated in FIG. 2 is one of many inteins that is bifunctional in that it mediates both protein splicing and DNA cleavage. This latter activity is mediated by the endonuclease domain (EN) 9 that interrupts the N- and C-terminal intein protein splicing domains 5, 7 illustrated in FIG. 2. Because the endonuclease activity is not required for protein splicing, split intein constructs capable of expressing cyclic peptides that are designed from such bifunctional inteins need not include the endonuclease domain (see, e.g., Wood et al., 1999, Nature Biotechnology 17:889-892). Thus, the precursor protein 50 illustrated in FIG. 4 does not include an endonuclease domain.

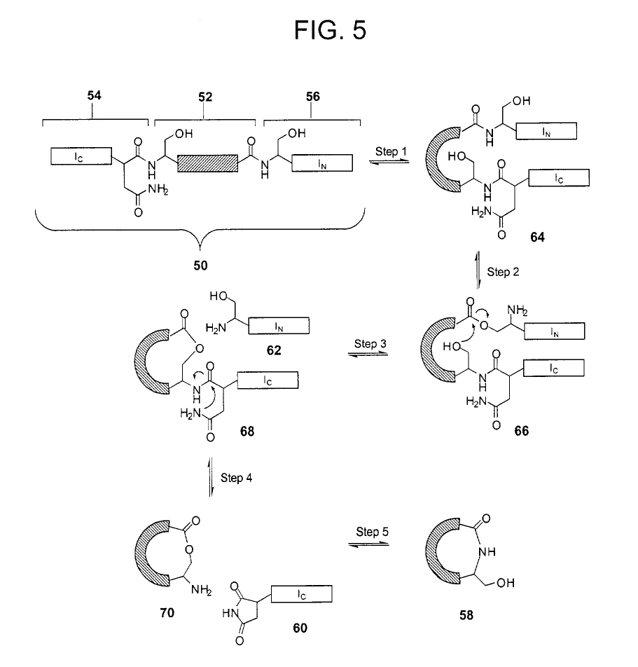

Referring again to FIG. 4, when such a precursor protein 50 is expressed in an appropriate host system, I_{C} 54 and I_{N} 56 physically come together to form an active intein (illustrated in FIG. 5) that catalyzes a protein splicing reaction that yields an extein cyclized in a head-to-tail fashion 58 and released C- and N-terminal intein domains 60 and 62. Like the splicing reaction catalyzed by the contiguous intein illustrated in FIGS. 2 and 3, the splicing reaction catalyzed by the split intein construct 50 of FIG. 4 also requires four nucleophilic displacements mediated by three conserved splice junction residues: (i) a Ser, Cys or Ala at the N-terminus of I_{N} 56; (ii) an Asn or Asp at the C-terminus of I_{C} 54; and (iii) a Ser, Cys or Thr at the N-terminus (beginning) of extein 52

The mechanism of the trans splicing reaction is illustrated in FIG. 5. In FIG. 5, the C-terminal Asn residue of I_{C} 54, the N-terminal Ser residue of extein 52 and the N-terminal Ser residue of I_{N} 56 are illustrated. Also, I_{C} 54 and I_{N} 56 are illustrated so as to highlight their coming together physically to constitute an active intein. Referring to FIG. 5, in Step 1, I_{C} 54 and I_{N} 56 come together to yield an active intein 64 in a conformation that forces extein 52 into a loop configuration. In Step 2, the N-terminal Ser of I_{N} 56 undergoes an O—C acyl migration to yield a reactive ester 66. In Step 3, the oxygen of the N-terminal Ser residue of extein 52 reacts with the ester to yield a lariat product 68 and released I_{N} 62. The active intein then resolves the lariat 68 via the formation of a succinimide that liberates a cyclized lactone form of the extein 70 and I_{C} 60. The lactone 70 then spontaneously rearranges to form the thermodynamically favored head-to-tail lactone form of the cyclic peptide 58.

The polynucleotides of the invention exploit this trans splicing ability of split inteins to yield polynucleotides capable of expressing cyclic peptide embodiments of the cyclic compounds of the invention that are cyclized in a head-to-tail fashion. Thus, the polynucleotides of the invention generally comprise a first segment encoding a C-terminal intein protein splicing domain (I_{C}), a second segment encoding a linear version of a cyclic peptide of the invention and a third segment encoding an N-terminal intein protein splicing domain (I_{N}). The three segments are arranged such that when expressed, the polynucleotide yields the precursor protein 50 (FIG. 4) in which extein 52 corresponds to the linear version of the desired cyclic peptide of the invention.

The polynucleotide may also encode or include additional elements, such as promoters operably linked to the portion of the polynucleotide encoding precursor protein 50 such that expression of precursor protein 50 is under the control of the promoter, reporter molecules such as fluorescent proteins, etc.

Nucleotide sequences that encode I_{C} and I_{N} may be derived from naturally-occurring split inteins (i.e., inteins that in nature are produced as two distinct polypeptide chains) or from contiguous inteins that have been artificially split and rearranged using known techniques. One example of a naturally-occurring split intein that may be used in connection with the polypeptides of the invention is Ssp DnaE (Wu et al., 1998, Proc. Natl. Acad. Sci. USA 95:9226). Another specific example is Ssp DnaB (Wu et al., 1998, Biochem Biophys Acta 1387:422-432 (PMID9748659); Evans et al., 1999, J. Biol. Chem. 274:18359-63 (PMID10373440)). A fairly comprehensive list of contiguous inteins that may be artificially split and rearranged according to the invention is published by New England Biolabs at http://www.neb.com/inteins/intreq.html. Guidance for splitting and rearranging such inteins for use in the polynucleotides of the invention may be found, for example, in WO 01/66565; Evans et al., 1999, J. Biol. Chem. 274:18359; Mills et al., 1998, Proc. Natl. Acad. Sci. USA 95:3543. Specific examples of contiguous inteins that may be split and rearranged in accordance with the invention include Psp-Pol-1 (Southworth et al., 1998, EMBO J. 17:918), Mycobacterium tuberculosis RccA intein (Lew et al., 1998, J. Biol. Chem. 273:15887; Shingledecker et al., 1998, Gene 207:187; Mills et al., 1998, Proc. Natl. Acad. Sci. USA 95:3543), Ssp/DnaB/MxeGyrA (Evans et al., 1999, J. Biol. Chem. 274:18359) and Pfu (Otoma et al., 1999, Biochemistry 38:16040; Yamazaki et al., 1998, J. Am. Chem. Soc. 120:5591). Additional specific examples are found in WO 01/66565.

Polynucleotides encoding precursor proteins including split intein domains that may be used to express cyclic peptide embodiments of the cyclic compounds of the invention, as well as expression vectors including such polynucleotides, methods for making such polynucleotides and cellular systems and conditions for expressing such polynucleotides to yield cyclic peptides are known in the art and are described, for example, in WO 01/66565, WO 00/36093; and the references cited. Any of these polynucleotides and expression systems may be routinely adapted to construct polynucleotides capable of expressing cyclic peptide embodiments of the cyclic compounds of the invention.

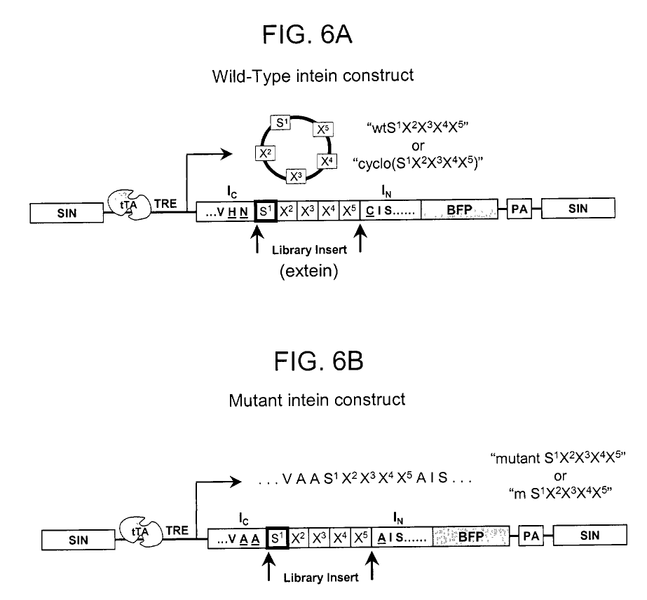

A specific example of a polynucleotide construct (in this case a retroviral construct) capable of expressing a cyclic peptide according to the invention is illustrated in FIG. 6A. Referring to FIG. 6A, the construct includes retroviral mutated non-functional LTR promoters (“SIN”) flanking an inverted intein (I_{C} and I_{N}). The illustrated amino acids of the intein are based upon the sequence of the inverted DnaB intein (Scott et al., 2001, Chem. Biol. 8:801-815). The I_{C} and I_{N} domains of the inverted intein flank the region encoding the cyclic peptide (library insert; extein). As discussed previously, inteins require nucleophilic residues after each splice junction for splicing activity at the junction (Mathys et al., 1999, Gene 231:1-13) and have an invariant Asn residue at the C-terminus of Ic. In FIG. 6A, the invariant Ser residue is boxed, and the required nucleophilic residues Cys, Asn and His of I_{N} and I_{C} are underlined. As will be explained in more detail in a later section, the construct of FIG. 6A was used in screening assays and also includes tetracycline-regulated elements (“TRE”) which respond to the tetracycline transactivator tTA-VP16 (“tTA”), a polyadenylation site (“PA”) and a region encoding blue fluorescent protein (“BFP”). The cyclic peptide expressed by this construct is delineated by the amino acids S¹X²X³X⁴X⁵, where “S” is serine and each “X” independently represents a genetically-encoded amino acid.

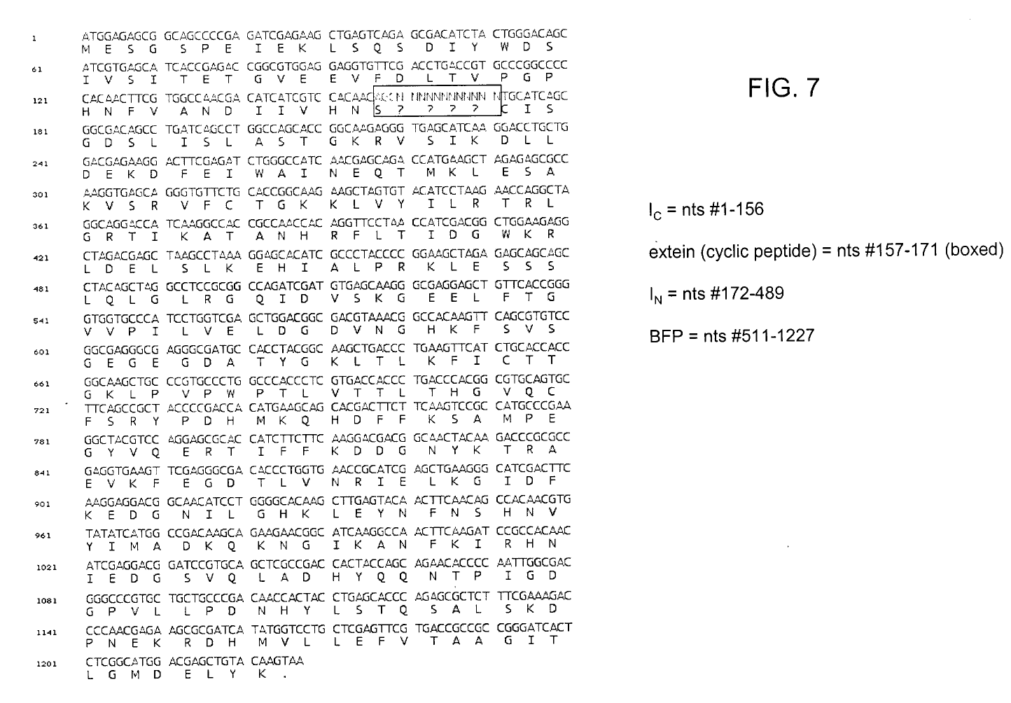

A specific example of a nucleotide sequence encoding the I_{C}-extein-I_{N} region of the construct of FIG. 6A is provided in FIG. 7. In FIG. 7, nucleotides #1-156 correspond to the I_{C} region of FIG. 6A, nucleotides #157-171 correspond to the cyclic peptide (extein nucleotides #147-149 encode a fixed Ser residue) region of FIG. 6A and nucleotides #172-489 correspond to the I_{C} region of FIG. 6A. Skilled artisans will recognize that in the cyclic peptide (extein) region of FIG. 7, “N” represents any base and that the length of this region may vary depending upon the number of residues comprising the desired cyclic peptide. The translated amino acids are shown below their respective codons. Stop codons are indicated with a period (“.”). Nucleotides #511-1227 encode BFP.

6.5 Methods of Making the Cyclic Compounds

6.5.1 Chemical Synthesis of the Cyclic Compounds

The cyclic compounds of the invention may be prepared using virtually any art-known technique for the preparation of cyclic peptides and cyclic peptide analogs. For example, the peptide analog may be prepared in linear or non-cyclized form using conventional solution or solid phase peptide and/or peptide analog syntheses and cyclized using standard chemistries. Preferably, the chemistry used to cyclize the compound will be sufficiently mild so as to avoid substantially degrading the compound. Suitable procedures for synthesizing the peptide and peptide analogs described herein, as well as suitable chemistries for cyclizing such compounds, are well known in the art.

For references related to synthesis of cyclic peptides the reader is referred to Tam et al., 2000, Biopolymers 52:311-332; Camamero et al, 1998, Angew. Chem. Intl. Ed. 37: 347-349; Tam et al., 1998, Prot. Sci. 7:1583-1592; Jackson et al., 1995, J. Am. Chem. Soc. 117:819-820; Dong et al., 1995, J. Am. Chem. Soc. 117:2726-2731; Ishida et al., 1995, J. Org. Chem. 60:5374-5375; WO 95/33765, published Jun. 6, 1995; Xue and DeGrado, 1994, J. Org. Chem. 60(4):946-952; Jacquier et al., 1991, In: Peptides 1990 221-222, Giralt and Andreu, Eds., ESCOM Leiden, The Netherlands; Schmidt and Neubert, 1991, In: Peptides 1990 214-215, Giralt and Andreu, Eds., ESCOM Leiden, The Netherlands; Toniolo, 1990, Int. J. Peptide Protein Res. 35:287-300; Ulysse et al., 1995, J. Am. Chem. Soc. 117:8466-8467; Durr et al., 1991, Peptides 1990 216-218, Giralt and Andreu, Eds., ESCOM Leiden, The Netherlands; Lender et al., 1993, Int. J. Peptide Protein Res. 42:509-517; Boger and Yohannes, 1990, J. Org. Chem. 55:6000-6017; Brady et al., 1979, J. Org. Chem. 4(18):3101-3105; Spatola et al., 1986, J. Am. Chem. Soc. 108:825-831; Seidel et al., 1991, In: Peptides 1990 236-237, Giralt and Andreu, Eds., ESCOM Leiden, The Netherlands; Tanizawa et al., 1986, Chem. Phar, Bull. 34(10):4001-4011; Goldenburg & Creighton, 1983, J. Mol. Biol. 165:407-413.

It is to be understood that the chemical linkage used to covalently cyclize the compounds of the invention need not be an amide linkage. Indeed, in many instances it may be desirable to modify the N- and C-termini of the linear or non-cyclized compound so as to provide, for example, reactive groups that may be cyclized under mild reaction conditions. Such linkages include, by way of example and not limitation amide, ester, thioester, CH₂—NH—, etc. Such cyclization reactions may be mediated by chemical cross-linking, chemical and/or enzymatic intramolecular ligation strategies. For example, Creighton and coworkers used a chemical cross-linking approach to prepare a head-to-tail cyclized version of bovine pancreatic trypsin inhibitor (Goldenberg et al., 1983, J. Mol. Biol. 165:407-413). Chemical (Camamero et al., 1998, Angew. Chem. Intl. Ed. 37:347-349; Tam et al., 1998, Prot. Sci. 7: 1583-1592; Camamero et al., 1997, Chem. Commun. 1997:1369-1380; Zhang & Tam, 1997, 119:2363-2370) and enzymatic (Jackson et al., 1995, J. Am. Chem. Soc. 117:819-820) intramolecular ligation methods have also been used to cyclize linear peptides under mild, aqueous conditions. These methods may be routinely adapted to synthesize the cyclic compounds of the invention.

Alternatively, linear forms at the cyclic compounds of the invention may be cyclized using an intramolecular version of the native chemical ligation approach described by Dawson et al., 1994, Science 266:776-779. Native chemical ligation involves the chemoselective reaction that occurs between an N-terminal Cys residue in one peptide or peptide analog and an α-thioester group with a second peptide or peptide analog, resulting in the formation of a peptide bond. Incorporation of both of these moieties into a single peptide or peptide analog leads to efficient head-to-tail cyclization (see, e.g., Camamero et al., 1983, supra; Tam et al., 1998, supra; Camamero et al., 1987, supra; Zhang & Tam, 1997, supra). Additional ligation chemistries and strategies that can be used to cyclize linear versions of the cyclic compounds of the invention are described in Tam et al., 2000, Biopolymers 52:311-322 and the references cited therein. Techniques and reagents for synthesizing peptides and peptide analogs having modified termini and chemistries suitable for cyclizing such modified peptides are well-known in the art.

Alternatively, in instances where the ends of the compounds are conformationally or otherwise constrained so as to make cyclization difficult, it may be desirable to attach linkers to the N- and/or C-termini to facilitate cyclization. Of course, it will be appreciated that such linkers will bear reactive groups capable of forming covalent bonds with the termini of the compound. Suitable linkers and chemistries are well-known in the art and include those previously described.

As will be readily appreciated by those having skill in the art, since the peptides and peptide analogs of the invention are cyclic, the designation of the N- and C-terminal amino acids is arbitrary. Thus, the compounds of the invention can be synthesized in linear or non-cyclized form starting from any amino acid residue. Preferably, the compounds of the invention are synthesized in a manner so as to provide a linear or non-cyclized compound that, when subjected to cyclization conditions, yields a substantial amount of cyclic compound. One of ordinary skill in the art will be able to choose an appropriate cyclization strategy for a particular compound sequence without undue experimentation.

Alternatively, the cyclic peptides and peptide analogs of the invention may be prepared by way of segment condensation. The preparation of both linear and cyclic peptides and analogs using segment condensation techniques is well-described in the art (see, e.g., Tam et al., 2000, supra; Liu et al., 1996, Tetrahedron Lett. 37(7):933-936; Baca, et al., 1995, J. Am. Chem. Soc. 117:1881-1887; Tam et al., 1995, Int. J. Peptide Protein Res. 45:209-216; Schnolzer and Kent, 1992, Science 256:221-225; Liu and Tam, 1994, J. Am. Chem. Soc. 116(10):4149-4153; Liu and Tam, 1994, Proc. Natl. Acad. Sci. USA 91:6584-6588; Yamashiro and Li, 1988, Int. J. Peptide Protein Res. 31:322-334).

Any disulfide linkages in the cyclic compounds of the invention may be formed before or after cyclization, but are preferably formed after cyclization. Formation of disulfide linkages, if desired, is generally conducted in the presence of mild oxidizing agents. Chemical oxidizing agents may be used, or the compounds may simply be exposed to atmospheric oxygen to effect these linkages. Various methods are known in the art, including those described, for example, by Tam, et al., 1979, Synthesis 955-957; Stewart et al., 1984, Solid Phase Peptide Synthesis, 2d Ed., Pierce Chemical Company Rockford, Ill.; Ahmed et al., 1975, J. Biol. Chem. 250:8477-8482; Pennington et al., 1991, Peptides 1990:164-166, Giralt and Andreu, Eds., ESCOM Leiden, The Netherlands. An additional alternative is described by Kamber et al., 1980, Helv. Chim. Acta 63:899-915. A method conducted on solid supports is described by Albericio, 1985, Int. J. Peptide Protein Res. 26:92-97. Any of these methods may be used to form disulfide linkages in the peptides of the invention.

6.5.2 Recombinant Synthesis

If the cyclic compound is composed entirely of gene-encoded amino acids, or a portion of it is so composed, the peptide or the relevant portion may also be synthesized using conventional recombinant genetic engineering techniques. In one embodiment, linear versions of the cyclic peptides are produced recombinantly and the resultant peptide cyclized or condensed, and optionally oxidized, as previously described to yield a cyclic peptide. In another embodiment, cyclic peptides are produced recombinantly utilizing polynucleotides capable of expressing a cyclic peptide, such as the polynucleotides described in Section 5.4.

For recombinant production, a polynucleotide sequence encoding a linear version of the peptide or a polynucleotide capable of expressing the cyclic peptide (for example, a polynucleotide encoding precursor protein 50 of FIG. 4) is inserted into an appropriate expression vehicle, i.e., a vector which contains the necessary elements for the transcription and translation of the inserted coding sequence, or in the case of an RNA viral vector, the necessary elements for replication and translation. The expression vehicle is then transfected into a suitable target cell which will express the peptide or cyclic peptide. Depending on the expression system used, the expressed peptide or cyclic peptide is then isolated by procedures well-established in the art. Methods for recombinant protein and peptide production are well known in the art (see, e.g., Sambrook et al., 1989, Molecular Cloning A Laboratory Manual, Cold Spring Harbor Laboratory, N.Y.; and Ausubel et al., 1989, Current Protocols in Molecular Biology, Greene Publishing Associates and Wiley Interscience, N.Y. each of which is incorporated by reference herein in its entirety.)

For linear peptides, to increase efficiency of production, the polynucleotide can be designed to encode multiple units of the peptide separated by enzymatic cleavage sites—either homopolymers (repeating peptide units) or heteropolymers (different peptides strung together) can be engineered in this way. The resulting polypeptide can be cleaved (e.g., by treatment with the appropriate enzyme) in order to recover the peptide units. This can increase the yield of peptides driven by a single promoter. In a preferred embodiment, a polycistronic polynucleotide can be designed so that a single mRNA is transcribed which encodes multiple peptides (i.e., homopolymers or heteropolymers) each coding region operatively linked to a cap-independent translation control sequence; e.g., an internal ribosome entry site (IRES). When used in appropriate viral expression systems, the translation of each peptide encoded by the mRNA is directed internally in the transcript; e.g., by the IRES. Thus, the polycistronic construct directs the transcription of a single, large polycistronic mRNA which, in turn, directs the translation of multiple, individual peptides. This approach eliminates the production and enzymatic processing of polyproteins and may significantly increase yield of peptide driven by a single promoter.

For cyclic peptides, production may be increased by designing the polypeptide to encode multiple repeats of the expressed precursor protein 50 (illustrated in FIG. 4). Such polynucleotide will express multiple copies of the encoded cyclic peptide. An example of a suitable construct for expressing multiple copies of a cyclic peptide is described in WO 00/36093 (see especially the disclosure at pages 22-23).

A variety of host-expression vector systems may be utilized to express the linear version of and the cyclic peptides described herein. These include, but are not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems.

The expression elements of the expression systems vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage lambda., plac, ptrp, ptac (ptrp-lac hybrid promoter) and the like may be used; when cloning in insect cell systems, promoters such as the baculovirus polyhedron promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the ³⁵S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5 K promoter) may be used; when generating cell lines that contain multiple copies of expression product, SV40-, BPV- and EBV-based vectors may be used with an appropriate selectable marker.

In cases where plant expression vectors are used, the expression of sequences encoding the peptides of the invention may be driven by any of a number of promoters. For example, viral promoters such as the ³⁵S RNA and 19S RNA promoters of CaMV (Brisson et al., 1984, Nature 310:511-514), or the coat protein promoter of TMV (Takamatsu et al., 1987, EMBO J. 6:307-311) may be used; alternatively, plant promoters such as the small subunit of RUBISCO (Coruzzi et al., 1984, EMBO J. 3:1671-1680; Broglie et al., 1984, Science 224:838-843) or heat shock promoters, e.g., soybean hspl7.5-E or hspl7.3-B (Gurley et al., 1986, Mol. Cell. Biol. 6:559-565) may be used. These constructs can be introduced into plant cells using Ti plasmids, Ri plasmids, plant virus vectors, direct DNA transformation, microinjection, electroporation, etc. For reviews of such techniques see, e.g. Weissbach & Weissbach, 1988, Methods for Plant Molecular Biology, Academic Press, N.Y., Section VIII, pp. 421-463; and Grierson & Corey, 1988, Plant Molecular Biology, 2d Ed., Blackie, London, Ch. 7-9.

In one insect expression system that may be used to produce linear versions and/or the cyclic peptides of the invention, Autographa californica, nuclear polyhidrosis virus (AcNPV) is used as a vector to express the foreign genes. The virus grows in Spodoptera frugiperda cells. A coding sequence may be cloned into non-essential regions (for example the polyhedron gene) of the virus and placed under control of an AcNPV promoter (for example, the polyhedron promoter). Successful insertion of a coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed (e.g., see Smith et al., 1983, J. Virol. 46:584; U.S. Pat. No. 4,215,051). Further examples of this expression system may be found in Current Protocols in Molecular Biology, Vol. 2, Ausubel et al., eds., Greene Publish. Assoc. & Wiley Interscience.

In mammalian host cells, a number of viral based expression systems may be utilized. In cases where an adenovirus is used as an expression vector, a coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing peptide in infected hosts. (e.g., See Logan & Shenk, 1984, Proc. Natl. Acad. Sci. USA 81:3655-3659). Alternatively, the vaccinia 7.5 K promoter may be used, (see, e.g. Mackett et al., 1982, Proc. Natl. Acad. Sci. USA 79:7415-7419; Mackett et al., 1984, J. Virol. 49:857-864; Panicali et al., 1982, Proc. Natl. Acad. Sci. USA 79:4927-4931).

Other expression systems for producing linear versions of and/or the cyclic peptides of the invention will be apparent to those having skill in the art.

6.5.3 Purification of Cyclic Compounds

The cyclic compounds of the invention can be purified by art-known techniques such as reverse phase chromatography high performance liquid chromatography, ion exchange chromatography, gel electrophoresis, affinity chromatography and the like. The actual conditions used to purify a particular compound will depend, in part, on synthesis strategy and on factors such as net charge, hydrophobicity, hydrophilicity, etc., and will be apparent to those having skill in the art.

For affinity chromatography purification, any antibody which specifically binds the compound may be used. For the production of antibodies, various host animals, including but not limited to rabbits, mice, rats, etc., may be immunized by injection with a compound. The compound may be attached to a suitable carrier, such as BSA, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette-Guerin) and Corynebacterium parvum.

Monoclonal antibodies to a compound may be prepared using any technique which provides for the production of antibody molecules by continuous cell lines in culture. These include, but are not limited to, the hybridoma technique originally described by Kohler & Milstein, 1975, Nature 256:495-497 and/or Kaprowski, U.S. Pat. No. 4,376,110; the human B-cell hybridoma technique described by Kosbor et al., 1983, Immunology Today 4:72 and/or Cote et al., 1983, Proc. Natl. Acad. Sci. USA 80:2026-2030; and the EBV-hybridoma technique described by Cole et al., 1985, Monoclonal Antibodies and Cancer Therapy, Alan R. Liss, Inc., pp. 77-96. In addition, techniques developed for the production of “chimeric antibodies” (Morrison et al., 1984, Proc. Natl. Acad. Sci. USA 81:6851-6855; Neuberger et al., 1984, Nature 312:604-608; Takeda et al., 1985, Nature 314:452-454; Boss, U.S. Pat. No. 4,816,397; Cabilly, U.S. Pat. No. 4,816,567) by splicing the genes from a mouse antibody molecule of appropriate antigen specificity together with genes from a human antibody molecule of appropriate biological activity can be used. Or “humanized” antibodies can be prepared (see, e.g., Queen, U.S. Pat. No. 5,585,089). Alternatively, techniques described for the production of single chain antibodies (see, e.g., U.S. Pat. No. 4,946,778) can be adapted to produce compound-specific single chain antibodies.

Antibody fragments which contain deletions of specific binding sites may be generated by known techniques. For example, such fragments include but are not limited to F(ab′)₂ fragments, which can be produced by pepsin digestion of the antibody molecule and Fab fragments, which can be generated by reducing the disulfide bridges of the F(ab′)₂ fragments. Alternatively, Fab expression libraries may be constructed (Huse et al., 1989, Science 246:1275-1281) to allow rapid and easy identification of monoclonal Fab fragments with the desired specificity for the peptide of interest.

The antibody or antibody fragment specific for the desired peptide can be attached, for example, to agarose, and the antibody-agarose complex is used in immunochromatography to purify peptides of the invention. See, Scopes, 1984, Protein Purification: Principles and Practice, Springer-Verlag New York, Inc., N.Y., Livingstone, 1974, Methods In Enzymology: Immunoaffinity Chromatography of Proteins 34:723-731.

6.6 Uses of the Cyclic Compounds

As discussed previously, the active cyclic compounds of the invention, can be used in a variety of in vitro and in vivo applications to regulate or modulate processes involved with the production and/or accumulation of IgE. For example, the active cyclic compounds can be used to modulate, and in particular inhibit, any or all of the following processes in vitro or in vivo: IgE production and/or accumulation; IL-4 induced switching of B-cells to produce IgE, IL-4 receptor-mediated IgE production; and IL-4 induced germline ε transcription. In a specific embodiment of the invention, the active cyclic compounds may be used to treat or prevent diseases characterized by, caused by or associated with production and/or accumulation of IgE. Such treatments may be administered to animals in veterinary contexts or to humans. Diseases that are characterized by, caused by or associated with IgE production and/or accumulation, and that can therefore be treated or prevented with the active cyclic compounds include, by way of example and not limitation, anaphylactic hypersensitivity or allergic reactions and/or symptoms associated therewith, atopic disorders such as atopic dermatitis, atopic eczema and atopic asthma, allergic rhinitis, allergic conjunctivitis, systemic mastocytosis, hyper IgE syndrome, IgE gammopathies and B-cell lymphoma.

When used to treat or prevent such diseases, the active cyclic compounds may be administered singly, as mixtures of one or more active cyclic compounds or in mixture or combination with other agents useful for treating such diseases and/or symptoms associated with such diseases. The active cyclic compounds may also be administered in mixture or in combination with agents useful to treat other disorders or maladies, such as steroids, membrane stabilizers, 5LO inhibitors, leukotriene synthesis and receptor inhibitors, IgE receptor inhibitors, β-agonists, tryptase inhibitors and antihistamines. The active cyclic compounds may be administered per se or as pharmaceutical compositions.

Pharmaceutical compositions comprising the active cyclic compounds of the invention may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the active cyclic compounds into preparations which can be used pharmaceutically. The actual pharmaceutical composition administered will depend upon the mode of administration. Virtually any mode of administration may be used, including, for example topical, oral, systemic, inhalation, injection, transdermal, etc.

The active cyclic compound may be formulated in the pharmaceutical compositions per se, or in the form of a pharmaceutically acceptable salt. As used herein, the expression “pharmaceutically acceptable salt” means those salts which retain substantially the biological effectiveness and properties of the active cyclic compound and which is not biologically or otherwise undesirable. Such salts may be prepared from inorganic and organic acids and bases, as is well-known in the art. Typically, such salts are more soluble in aqueous solutions than the corresponding free acids and bases.

For topical administration, the active cyclic compound(s) may be formulated as solutions, gels, ointments, creams, suspensions, etc. as are well-known in the art.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active cyclic compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives.

Alternatively, the injectable formulation may be provided in powder form for reconstitution with a suitable vehicle, including but not limited to sterile pyrogen free water, buffer, dextrose solution, etc., before use. To this end, the active cyclic compound(s) may dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active cyclic compound.

For buccal administration, the compositions may take the form of tablets or lozenges formulated in conventional manner.

For rectal and vaginal routes of administration, the active cyclic compound(s) may be formulated as solutions (for retention enemas) suppositories or ointments containing conventional suppository bases such as cocoa butter or other glycerides.

For administration by inhalation, the active cyclic compound(s) can be conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

For prolonged delivery, the active cyclic compound(s) can be formulated as a depot preparation, for administration by implantation; e.g., subcutaneous, intradermal, or intramuscular injection. Thus, for example, the active ingredient may be formulated with suitable polymeric or hydrophobic materials (e.g., as an emulsion in an acceptable oil) or ion exchange resins, or as sparingly soluble derivatives; e.g., as a sparingly soluble salt.

Alternatively, transdermal delivery systems manufactured as an adhesive disc or patch which slowly releases the active cyclic compound(s) for percutaneous absorption may be used. To this end, permeation enhancers may be used to facilitate transdermal penetration of the active cyclic compound(s). Suitable transdermal patches are described in for example, U.S. Pat. No. 5,407,713.; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other pharmaceutical delivery systems may be employed. Liposomes and emulsions are well-known examples of delivery vehicles that may be used to deliver active cyclic compounds(s). Certain organic solvents such as dimethylsulfoxide (DMSO) may also be employed, although usually at the cost of greater toxicity.

The pharmaceutical compositions may, if desired, be presented in a pack or dispenser device which may contain one or more unit dosage forms containing the active cyclic compound(s). The pack may, for example, comprise metal or plastic foil, such as a blister pack. The pack or dispenser device may be accompanied by instructions for administration.

6.6.1 Gene Therapy

As will be recognized by skilled artisans, active cyclic compound(s) that are peptides composed wholly of gene-encoded amino acids may be administered utilizing well-known gene therapy techniques. According to such techniques, a polynucleotide capable of expressing a cyclic peptide of the invention, such as a polynucleotide of the invention described supra, may be introduced either in vivo, ex vivo, or in vitro in a viral vector. Such vectors include an attenuated or defective DNA virus, such as but not limited to, herpes simplex virus (HSV), papillomavirus, Epstein Barr virus (EBV), adenovirus, adeno-associated virus (AAV), and the like. Defective viruses, which entirely or almost entirely lack viral genes, are preferred.

Defective virus is not infective after introduction into a cell. Use of defective viral vectors allows for administration to cells in a specific, localized area, without concern that the vector can infect other cells. For example, in the treatment of the various diseases described herein, lymphocyte B-cells can be specifically targeted. Examples of particular vectors include, but are not limited to, a defective herpes virus I (HSV1) vector (Kaplitt et al., 1991, Molec. Cell. Neurosci. 2:320-330), an attenuated adenovirus vector, such as the vector described by Stratford-Perricaudet et al., 1992, J. Clin. Invest. 90:626-630 and a defective adeno-associated virus vector (Samulski et al., 1987, J. Virol. 61:3096-3101; Samulski et al., 1989, J. Virol. 63:3822-3828).

Preferably, for in vitro administration, an appropriate immunosuppressive treatment is employed in conjunction with the viral vector, e.g., adenovirus vector, to avoid immuno-deactivation of the viral vector and transfected cells. For example, immunosuppressive cytokines, such as interleukin-12 (IL-12), interferon-γ(IFN-γ), or anti-CD4 antibody, can be administered to block humoral or cellular immune responses to the viral vectors (see, e.g., Wilson, 1995, Nat. Med. 1(9):887-889). In addition, it is advantageous to employ a viral vector that is engineered to express a minimal number of antigens.

In another embodiment the gene can be introduced in a retroviral vector, e.g., as described in Anderson et al., U.S. Pat. No. 5,399,346; Mann et al., 1983, Cell 33:153; Temin et al., U.S. Pat. No. 4,650,764; Temin et al., U.S. Pat. No. 4,980,289; Markowitz et al., 1988, J. Virol. 62:1120 (1988); Temin et al., U.S. Pat. No. 5,124,263; Dougherty et al., WO 95/07358; and Kuo et al., 1993, Blood 82:845. Targeted gene delivery is described in WO 95/28494.

Alternatively, the vector can be introduced by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker (Felgner et. al., 1987, Proc. Natl. Acad. Sci. USA 84:7413-7417; Mackey et al., 1988, Proc. Natl. Acad. Sci. USA 85:8027-8031). The use of cationic lipids may promote encapsulation of negatively charged nucleic acids, and also promote fusion with negatively charged cell membranes (Felgner & Ringold, 1989. Science 337:387-388). The use of lipofection to introduce exogenous genes into the specific organs in vivo has certain practical advantages. Molecular targeting of liposomes to specific cells represents one area of benefit. It is clear that directing transfection to particular cell types would be particularly advantageous in a tissue with cellular heterogeneity, such as pancreas, liver, kidney, and the brain. Lipids may be chemically coupled to other molecules for the purpose of targeting (see Mackey et al., 1988, supra). Targeted peptides, e.g., hormones or neurotransmitters, and proteins such as antibodies, or non-peptide molecules could be coupled to liposomes chemically.

It is also possible to introduce the vector as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, e.g., Wu et al., 1992, J. Biol. Chem. 267:963-967; Wu & Wu, 1988, J. Biol. Chem., 263:14621-14624; Canadian Patent Application No. 2,012,311).

Naked nucleic acids capable of expressing the active cyclic peptide may also be introduced using the gene-activated matrices described, for example, in U.S. Pat. No. 5,962,427.

6.7 Effective Dosages

The active cyclic compound(s) of the invention, or compositions thereof, will generally be used in an amount effective to treat or prevent the particular disease being treated. The compound(s) may be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. For example, administration of an active cyclic compound to a patient suffering from an allergy provides therapeutic benefit not only when the underlying allergic response is eradicated or ameliorated, but also when the patient reports a decrease in the severity or duration of the symptoms associated with the allergy following exposure to the allergan. Therapeutic benefit also includes halting or slowing the progression of the underlying disease or disorder, regardless of whether improvement is realized.

For prophylactic administration, the active compound may be administered to a patient at risk of developing a disorder characterized by, caused by or associated with IgE production and/or accumulation, such as the various disorders previously described. For example, if it is unknown whether a patient is allergic to a particular drug, the active compound may be administered prior to administration of the drug to avoid or ameliorate an allergic response to the drug. Alternatively, prophylactic administration may be applied to avoid the onset of symptoms in a patient diagnosed with the underlying disorder. For example, an active compound may be administered to an allergy sufferer prior to expected exposure to the allergan. Active compounds may also be administered prophylactically to healthy individuals who are repeatedly exposed to agents known to induce an IgE-related malady to prevent the onset of the disorder. For example, an active compound may be administered to a healthy individual who is repeatedly exposed to an allergan known to induce allergies, such as latex, in an effort to prevent the individual from developing an allergy.

The amount of active compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, etc. Determination of an effective dosage is well within the capabilities of those skilled in the art.

Initial dosages may be estimated from in vitro assays. For example, an initial dosage for use in animals may be formulated to achieve a circulating blood or serum concentration of active compound that inhibits about 25% or more of IL-4 induced IgE production, or a process associated therewith, such as germline ε transcription, as measured in an in vitro assay. Calculating dosages to achieve such circulating blood or serum concentrations taking into account the bioavailability of the particular active compound is well within the capabilities of skilled artisans. For guidance, the reader is referred to Fingl & Woodbury, “General Principles,” In: The Pharmaceutical Basis of Therapeutics, Chapter 1, pp. 1-46, 1975, and the references cited therein.

Initial dosages can also be estimated from in vivo data, such as animal models. Animals models useful for testing the efficacy of compounds to treat or prevent diseases characterized by, caused by or associated with IgE production and/or accumulation are well-known in the art. Ordinarily skilled artisans can routinely adapt such information to determine dosages suitable for human administration.

Dosage amounts will typically be in the range of from about 1 mg/kg/day to about 100 mg/kg/day, 200 mg/kg/day, 300 mg/kg/day, 400 mg/kg/day or 500 mg/kg/day, but may be higher or lower, depending upon, among other factors, the activity of the active compound, its bioavailability, the mode of administration and various factors discussed above. Dosage amount and interval may be adjusted individually to provide plasma levels of the active compound(s) which are sufficient to maintain therapeutic or prophylactic effect. In cases of local administration or selective uptake, such as local topical administration, the effective local concentration of active compound(s) may not be related to plasma concentration. Skilled artisans will be able to optimize effective local dosages without undue experimentation.

The compound(s) may be administered once per day, a few or several times per day, or even multiple times per day, depending upon, among other things, the indication being treated and the judgement of the prescribing physician.

Preferably, the active compound(s) will provide therapeutic or prophylactic benefit without causing substantial toxicity. Toxicity of the active compound(s) may be determined using standard pharmaceutical procedures. The dose ratio between toxic and therapeutic (or prophylactic) effect is the therapeutic index. Active compound(s) that exhibit high therapeutic indices are preferred.

6.8 Other Uses

While the cyclic compounds of the invention find particular use in the various in vitro, in vivo and therapeutic contexts described herein, skilled artisans will recognize that the compounds have additional uses. For example, the cyclic compounds may be used in screening or other assays to identify binding partners or targets involved in the IL-4 signal transduction pathway leading to isotype switching of B-cells to produce IgE.

The invention having been described, the following examples are offered by way of illustration and not limitation.

### 7. EXAMPLES

7.1 Identification of Cyclic Peptides SEQ ID NOS:1-20 from Random Libraries of Cyclic Peptides

Cyclic peptides SEQ ID NOS:1-20 were identified by screening retrovirally encoded cyclic peptide libraries of 4-, 5-, 6- and 7-mers for the ability to inhibit IL-4 induced germline ε transcription using the HBEGF2a-GFP dual reporter phenotypic screening system described in WO 01/31232. To construct the random libraries, A5T4 reporter cells (described in more detail below) were infected with a cyclic peptide library (prepared as described in WO 97/27213 using the intein constructs described in WO 01/66565; see also WO 01/34806 at page 39, line 36 through page 40, line 19). The retroviral vector used includes a reporter gene encoding blue fluorescent protein (BFP) fused upstream of the region encoding the precursor protein via a linker region encoding an α-helical peptide linker. The library was constructed in the pTRA retroviral vector, which rendered expression of the viral insert, and hence expression of the BFP-peptide product, tetracycline (Tet) or doxycycline (Dox) dependent (Lorens et al., 2000, Mol. Ther. 1:438-47). The BFP reporter gene provides a rapid phenotypic assay to determine whether cells were infected: infected cells express BFP-peptide and fluoresce blue (phenotype BFP⁺); uninfected cells do not express BFP-peptide, and do not fluoresce blue (phenotype BFP⁻). To reduce the number of stop codons, the region of the vector encoding the random portion of the cyclic peptide was of the sequence (NNK)_{3, 4, 5 or 6}, where each N independently represents A, T, C or G and each K independently represents T or G. The library was also biased to account for degeneracy in the genetic code. A cartoon illustrating the features of the retroviral vector is provided in FIG. 6A. The sequence of the coding strand of a portion of this retroviral vector is provided in FIG. 7.

To establish the A5T4 reporter cell line, BJAB cells were infected with a retroviral vector containing an IL-4 responsive 600 bp fragment of the epsilon promoter (Pε) followed by a fusion nucleic acid encoding HBEGF-2a-GFP and a polyadenylation sequence cloned in the reverse orientation into pCGSin (Lorens et al., 2000, Mol. Ther. 1:438-447). Infected cells were induced with IL-4 and single cell sorted by FACS for GFP reporter expression. One of the cell clones identified with the desired profile was designated A5. This cell line was infected with the CtTAIH retroviral vector expressing tTA-VP16 ires hygromycin phosphotranferase gene (Lorens et al., 2000, Virology 272:7) selected with hygromycin and single cell cloned. The clone selected for the screening cell line was characterized for tetracycline regulation and was designated A5T4.

Owing to the features of the A5T4 reporter cell line, cyclic peptides that block IL-4 signaling block the expression of HBEGF and survive diphtheria killing selection. GFP is used to monitor IL-4 induction of Pε by GFP fluorescence. The clearing site ribosomal-skip 2a sequence (Ryan et al., 1991, J. Gen. Virol. 72 (Part 11):2727-2732), which is positioned between HBEGF and GFP, allows the release and optimal function of GFP from HBEGF. Thus, when expressed, the dual function reporter cleaves into two pieces: a heparin-binding epidermnal growth factor-like growth factor (HBEGF) and a green fluorescent protein (GFP). In this reporter system, cells ectopically expressing HBEGF are capable of translocating diphtheria toxin (DT) into their cytoplasm, leading to rapid, acute cytotoxicity. Cells that do not express HBEGF are spared this fate and continue to survive even in the presence of high concentrations of DT.

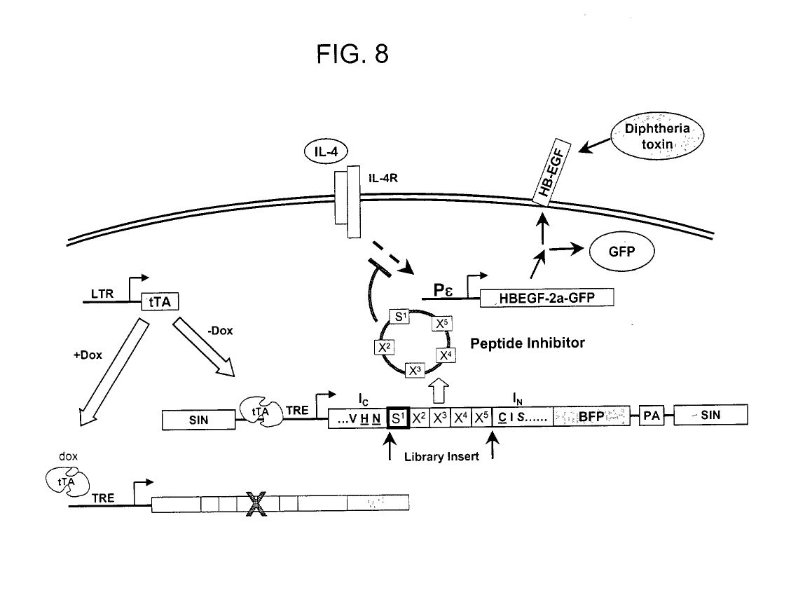

The A5T4 reporter cell line was further engineered to express the tetracycline-regulated transactivator tTA-VP16 (Gossen et al., 1995, Science 268:1766-69), allowing for regulation of peptide library expression with Tet or Dox through the tetracycline responsive elements (TRE) in the cyclic peptide library construct. In the presence of Tet or Dox (+Dox), expression of the peptide is turned off. Conversely, in the absence of Tet or Dox (−Dox), expression of the peptide is turned on. Thus, according to this dual phenotypic reporter system, unstimulated (−IL-4) control cells expressing a random cyclic peptide fluoresce blue (BFP⁺). Following stimulation with IL-4 (+IL-4) or another IL-4 effector, BFP⁺ cells expressing a non-inhibitory cyclic peptide fluoresce green and, in addition are sensitive to DT. Stimulated BFP⁺ cells expressing an inhibitory cyclic peptide do not fluoresce green and are not DT sensitive. The toxin-conditional selection and Tet or Dox-controlled peptide expression features of the A5T4 screening line are illustrated in FIG. 8.

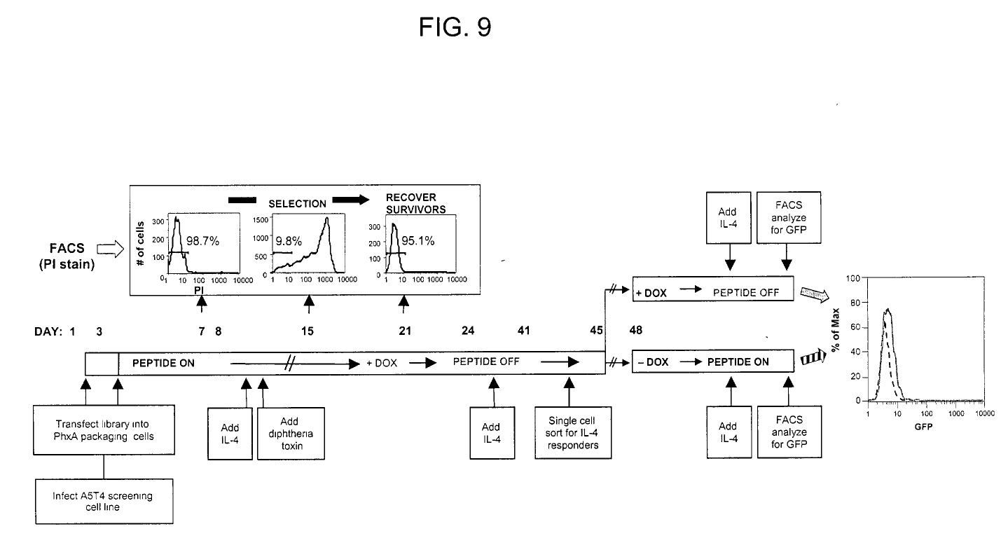

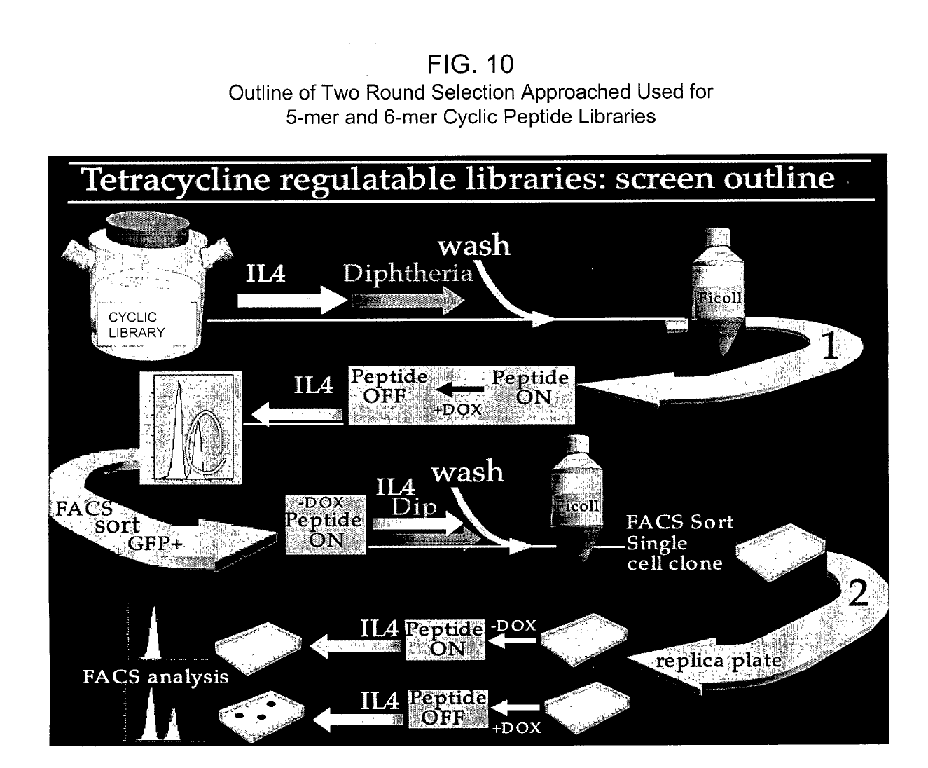

The functional selection and screening strategy, outlined in FIGS. 9 and 10, takes advantage of the versatility of the A5T4 cell line. Briefly, A5T4 cells infected with the retroviral intein library illustrated in FIG. 6A were stimulated with IL-4 (30 u/ml) to cause transcription of HBEGF and subsequently treated with diphtheria toxin (20 ng/ml). Most cells died and the surviving cells were recovered by day 21. The surviving cells represented mainly a background population of cells that failed to respond to IL-4 due to various epigenetic factors (false positives) and a minority of cells that were prevented from responding to IL-4 by the peptides (putative inhibitors). Returning to FIG. 9, FACS profiles of cell populations stained with propidium iodide (“PI”) at days 7, 15 and 21 are shown in the inset above the timeline (gating indicates live cell population). Day 21 marks the recovery of the cells surviving DT selection. The rare cells being regulated by the putative inhibitors were further enriched by treating them with Dox to repress cyclic peptide expression prior to a second round of IL-4 stimulation. With the peptide turned off, those cells responding to IL-4 stimulation as monitored by their GFP expression were FACS sorted and single cell cloned. Finally, the single cell clones were replica plated, grown in the presence or absence of Dox, stimulated with IL-4 and reanalyzed by FACS for GFP fluorescence. Single cell clones containing inhibitory peptides were selected based on inhibition of GFP fluorescence in the absence of Dox (−Dox). A representative GFP FACS profile for a Dox-regulatable clone is shown to the right of the timeline. The larger libraries (i.e., the 6- and 7-mer libraries) underwent two rounds of selection before single cell cloning, i.e., they were bulk sorted after the first round of selection then single cell cloned after the second round of selection, as illustrated in FIG. 10.

Referring to FIG. 9 or 10, after single cell clones had expanded, cells were replica-plated and grown in the presence or absence of 100 ng/ml Dox for 4 days. Both plates were then stimulated with IL-4 (30 U/ml) and, after 3 more days, both populations were analyzed by FACS to measure GFP fluorescence. FACS data were converted to reporter ratios, which are defined as the ratio of the geometric mean of GFP fluorescence of the +IL-4/+Dox (peptide off) population over the geometric mean of the +IL-4/−Dox (peptide on) population. Cells expressing a cyclic peptide that inhibits germline ε transcription have reporter ratios of >1.1, typically >1.12, >1.13, >1.14 or >1.15.

56 out of 1552 clones tested showed Dox-regulated inhibition of the ε-promoter driven reporter. That is, a decrease in IL-4 induced GFP reporter expression in the absence of Dox relative to the GFP expression in the presence of Dox. The Dox regulation of Pε driven transcription strongly suggests that the retroviral inserts were responsible for the ε-promoter inhibition.

7.2 The Clones Transfer Their Phenotypes into Naive Cells

To confirm peptide-mediated inhibition of IL-4 stimulated Ps-driven transcription, the library insert cDNAs from the 56 Dox-regulated clones were rescued by real time PCR (“RT-PCR”), cloned into the parental retroviral vector and re-introduced into naive A5T-4 cells. Of the 56 clones, 34 were successfully transferred. RT-PCR was performed using Titan RT-PCR kit ((Boehringer Mannheim, Germany) with 5′-CGTTTCTGATAGGCACCTATTGGTC-3′ (SEQ ID NO:48) as the reverse primer. Cycle conditions were 50° C. for 30 minutes, and 94° C. for 2 minutes. This was followed by 40 cycles, each consisting of 94° C. for 30 seconds, 60° C. for 30 seconds and 72° C. for 45 seconds and completed with a final extension of 10 minutes at 72° C. The resulting PCR reaction was cleaned using a Qiaquick column (Qiagen PCR, Qiagen, Valencia, Calif.). Sequential restriction enzyme cuts were made using MluI at 37° C. followed by BclI (both from New England Biolabs, Beverly, Mass.) at 50° C. Digests were run on 2% agarose gels and purified using a Qiaquick column. Ligations were done using standard ligation methods for T4 DNA ligase (New England Biolabs). The ligation was then transformed into Top10 chemically competent cells (Invitrogen, St. Louis, Mo.) and plated on LBtamp agar media. Individual colonies were picked and DNA was isolated and sequenced.

Phoenix packaging cells were transfected with the retroviral vector illustrated in FIG. 6A encoding a particular cyclic peptide as described in WO 97/27213. Naïve A5T4 cells were infected with the resultant retrovirons and grown for three days. The infected cells (BFP⁺) were stimulated with IL-4 (30 U/ml) and, after three days, were assessed by FACS for BFP and GFP fluorescence. In this assay, there were two populations of cells: cells that express BFP (BFP⁺) and cells that do not (BFP⁻).

FIG. 11, Panel A, shows the effect one cyclic peptide, cyclo(SRVEI), on PE-driven expression of HBEGF-2a-GFP. The BFP FACs profile is shown on the left, with the peaks for the BFP⁺ and BFP⁻ populations labeled. In the absence of IL-4 (top GFP FACs profile), both the infected population (BFP⁺) and the uninfected population (BFP⁻) showed the same basal level of GFP fluorescence. In the presence of IL-4 (bottom GFP FACs profile), the GFP fluorescence of the infected population (BFP⁺) was significantly lower than that of the uninfected population (BFP⁻). Based on the net increase of geometric mean GFP fluorescence following IL-4 stimulation, expression of the cyclo(SRVEI) peptide caused 51% inhibition of ε-promoter induction as compared to the uninfected population.

The GFP fluorescence data corresponding to the BFP⁻/BFP⁺in the presence of IL-4 stimulation were obtained for 34 clones and reporter ratios were calculated therefrom. Of the 34 clones tested in naive A5T4 cells, 33 were unique, of which 20 were found to strongly inhibit ε transcription. Three others had reporter wt ratios in the range of about 1.12-1.2. The names of the clones, sequences of the regions of the polynucleotides encoding the cyclic peptides SEQ ID NOS:1-23, the sequences of the expressed cyclic peptides SEQ ID NOS:1-23 and their respective reporter ratios are provided in TABLE 1, below (“wild-type” reporter ratios; each reporter ratio is the average of 3 values):

TABLE 1

| | | | | | Wild-Type | Mutant |
|---|---|---|---|---|---|---|
| | | | | | Reporter Ratio | Reporter Ratio |
| Clone Name | Peptide Sequence | Peptide SEQ ID NO | Nucleic Acid (NA) Sequence | NA SEQ ID NO | (± std) | (± std) |
| L6-4 PL#4 F9-B. | cyclo(S F V T W) | cyclo(SEQ ID NO:1) | AGCTTTGTTACTTGG | (SEQ ID NO:24) | 1.46 ± 0.12 | 1.25 ± 0.06 |
| L6-4 PL#5 A4-B. | cyclo(S R V E I) | cyclo(SEQ ID NO:2) | AGCCGGGTGGAGATT | (SEQ ID NO:25) | 1.53 ± 0.16 | 1.06 ± 0.06 |
| L6-4 PL#7 B3-B. | cyclo(S A R F V) | cyclo(SEQ ID NO:3) | AGCGCGAGGTTTGTT | (SEQ ID NO:26) | 1.52 ± 0.04 | 1.43 ± 0.03 |
| L6-4 PL#3 C7-B. | cyclo(S L N R I) | cyclo(SEQ ID NO:4); | AGCCTTAATCGGATT | (SEQ ID NO:27) | 1.45 ± 0.07 | 1.40 ± 0.08 |
| L6-4 PL#4 F9-C. | cyclo(S Y F T S C W) | cyclo(SEQ ID NO:5) | AGCTATTTTACTTCTTGTTGG | (SEQ ID NO:28) | 1.36 ± 0.07 | 1.27 ± 0.01 |
| L6-4 PL#6 D7-B. | cyclo(S S L R W) | cyclo(SEQ ID NO:6) | AGCTCGCTGAGGTGG | (SEQ ID NO:29) | 1.31 ± 0.05 | 1.21 ± 0.02 |
| L6-4 PL#3 B5-C. | cyclo(S F G R S) | cyclo(SEQ ID NO:7) | AGCTTTGGTCGTTCG | (SEQ ID NO:30) | 1.20 ± 0.02 | 1.08 ± 0.06 |
| E7-3 PL#1 A3-C. | cyclo(S E M F S I Q) | cyclo(SEQ ID NO:8) | AGCGAGATGTTTTCGATTCAG | (SEQ ID NO:31) | 1.28 ± 0.03 | 1.13 ± 0.03 |
| E7-3 PL#1 E14-C. | cyclo(S R N W S H) | cyclo(SEQ ID NO:9) | AGCCGTAATTGGTCGCAT | (SEQ ID NO:32) | 1.18 ± 0.04 | 1.06 ± 0.04 |
| L6-4 PL#6 F1-C. | cyclo(S N F T F) | cyclo(SEQ ID NO:10) | AGCAATTTTACGTTT | (SEQ ID NO:33) | 1.28 ± 0.05 | 1.08 ± 0.05 |
| L6-4 PL#9 G3-B. | cyclo(S N I P Q) | cyclo(SEQ ID NO:11) | AGCAATATTCCGCAG | (SEQ ID NO:34) | 1.20 ± 0.01 | 1.10 ± 0.02 |
| L6-4 PL#18 D4-A. | cyclo(S G A D S) | cyclo(SEQ ID NO:12) | AGCGGGGCGGATTCG | (SEQ ID NO:35) | 1.23 ± 0.02 | 1.08 ± 0.05 |
| L6-4 PL#6 G8-B. | cyclo(S V I E Q) | cyclo(SEQ ID NO:13) | AGCGTTATTGAGCAG | (SEQ ID NO:36) | 1.22 ± 0.04 | 1.14 ± 0.04 |
| L6-3 PL#1 A9-B. | cyclo(S Y S Q) | cyclo(SEQ ID NO:14) | AGCTATAGTCAG | (SEQ ID NO:37) | 1.18 ± 0.01 | 1.07 ± 0.01 |
| L6-4 PL#16 H2-B. | cyclo(S R R D R I) | cyclo(SEQ ID NO:15) | AGCAGGCGGGATCGTATT | (SEQ ID NO:38) | 1.31 ± 0.03 | 1.08 ± 0.04 |
| L6-4 PL#13 F8-A. | cyclo(S T G P R) | cyclo(SEQ ID NO:16) | AGCACGGGTCCTCGT | (SEQ ID NO:39) | 1.19 ± 0.03 | 1.18 ± 0.04 |
| L6-4 PL#6 G12-B. | cyclo(S V V T R) | cyclo(SEQ ID NO:17) | AGCGTTGTGACTCGG | (SEQ ID NO:40) | 1.19 ± 0.03 | 1.26 ± 0.03 |
| E7-3 PL#1 E14-A. | cyclo(S P W K L V G) | cyclo(SEQ ID NO:18) | AGCCCTTGGAAGTTGGTTGGG | (SEQ ID NO:41) | 1.27 ± 0.03 | 1.02 ± 0.06 |
| L6-4 PL#3 B5-B. | cyclo(S W A Q A) | cyclo(SEQ ID NO:19) | AGCTGGGCGCAGGCT | (SEQ ID NO:42) | 1.25 ± 0.02 | 1.10 ± 0.04 |
| L6-4 PL#1 E6-C. | cyclo(S D H S Q) | cyclo(SEQ ID NO:20) | AGCGATCATTCGCAG | (SEQ ID NO:43) | 1.20 ± 0.04 | 1.06 ± 0.06 |
| L6-4 PL#18 C11-B. | cyclo(S S C M R) | cyclo(SEQ ID NO:21) | AGCTCTTGTATGCGT | (SEQ ID NO:44) | 1.12 ± 0.03 | 1.20 ± 0.12 |
| E7-3 PL#1 A3-A. | cyclo(S S F T) | cyclo(SEQ ID NO:22) | AGCTCGTTTACG | (SEQ ID NO:45) | 1.10 ± 0.03 | 1.05 ± 0.04 |
| L6-4 PL#4 F6-C. | cyclo(S R R H C C H) | cyclo(SEQ ID NO:23) | AGCCGTCGGCATTGTTGTCAT | (SEQ ID NO:46) | 1.09 ± 0.03 | 1.02 ± 0.03 |

7.3 Inhibitory Activity Is Due to Expressed Cyclic Peptide

As discussed previously, intein splicing is dependent upon the presence of nucleophilic residues at the splice junctions (underlined in FIG. 6A; see Perler & Adam, 2000, Curr. Opin. Biotechnol. 11:377-383). To confirm that the inhibitory activity of the clones of TABLE 1 was due to the expression of a cyclic peptide, mutants were created that lacked the ability to catalyze the splicing reaction due to the replacement of these three critical underlined nucleophilic residues with alanines. These mutants (illustrated in FIG. 6B) were then used to infect naive A5T4 cells as described above.

The three alanines were introduced using double-stranded adapters: Adapter 1: 5′PHOS-TCGTCGCCCGCCAGCXXXXXXXXXXGCCATCAGCGGCGACAGCCT-3′ (SEQ ID NO:49) Adapter 2: 5′PHOS-GATCAGGCTGTCGCCGCTGATGGCXXXXXXXXXXXXGCTGGCGGCGACGATG-3′ (SEQ ID NO:50). The second adapter is flanked by a built in DrdI site (5 prime) and a Bell site (3 prime). Adapters were annealed by mixing equimolar ratios, heating to 95° C. and slow cooling to room temperature. Annealed adapters were gel purified and ligated by standard methods into the parent library intein vector DnaBO e-BFP (ACUC)* cut with DrdI and Bell. The ligation was then transformed into Top10 chemically competent cells (Invitrogen, St. Louis, Mo.) and plated on LB+amp agar media. Individual colonies were picked and DNA was isolated and sequenced. (See also Mathys et al., 1999, Gene 231:1-13).

A representative FACS profile for a mutant intein of FIB. 6B expressing the peptide SRVEI is shown in FIG. 11, Panel B. No significant difference is observed between the BFP+ (peptide on) and BFP− (peptide off) populations in either IL-4 unstimulated or stimulated conditions. The GFP reporter ratios of the 23 mutant clones tested are provided in TABLE 1, supra.

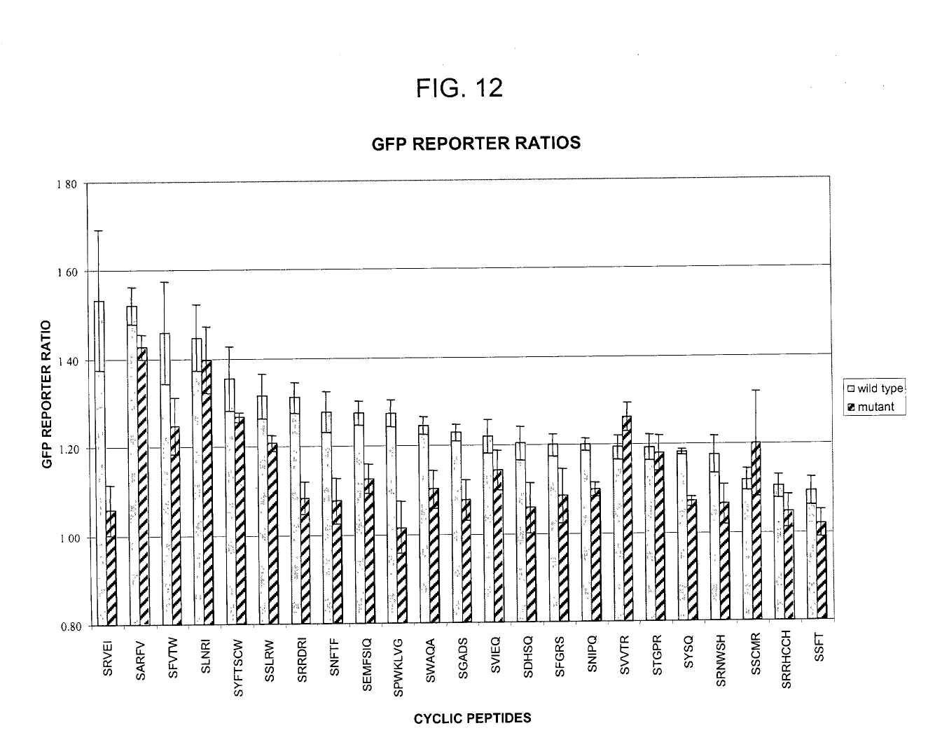

The GFP reporter ratios of the mutant inteins, as compared with the wild-type intein constructs, are presented in FIG. 12. Twelve of the active peptides (SRVEI, SRRDRI, SNFTF, SEMFSIQ, SPWKLVG, SWAQA, SGADS, SDHSQ, SFGRS, SNIPQ, SYSQ, SRNWSH), when presented in the mutant intein context, became inactive (not effective in blocking epsilon promoter transcription). Five of the active peptides (SARFV, SFVTW, SYFTSCW, SSLRW, SVIEQ) demonstrated a decrease in activity, and three active peptides (SLNRI, SVVTR, STGPR) remained active when presented in the mutant intein context. Analogous to peptides presented by other protein scaffolds (Peelle et al., 2001, Chem. Biol. 8:521-534), these last eight peptides may also be active when presented by the intein structure as a display scaffold. It is not known whether these peptides were active both as cyclic peptides and in the fused context with the intein protein.

7.4 Cyclic Peptides SEQ ID NOS:1-20 Inhibit Transcription of an Endogenous Germline ε Promoter

The ability of cyclic peptides SEQ ID NOS:1-20 to inhibit transcription of an endogenous germline ε promoter was confirmed using quantitative real time PCR (Taqman®; Applied Biosystems, Foster City, Calif.). Briefly, A5T4 cells were infected with retrovirus capable of expressing the cyclic peptides (prepared as described above). Cells were first sorted for BFP to ensure that they were retrovirally infected. Several different assay conditions were tested: −Dox/-IL-4, −Dox/+IL-4, +Dox/-IL-4, +Dox/+IL-4. All experiments were conducted in triplicate. BFP positive sorted cells were thereafter maintained in Dox (peptide off). To obtain the −Dox condition, cells were washed 2× with media and maintained in Dox-free culture for 72 hours. Cells in +/−Dox were split to 50,000 cells per ml. The following day, IL-4 was added (30 units/ml) and cells were incubated for 3 days before FACS analysis and pelleting for RNA extraction. The primers and probe used were as follows (the probe was labeled at the 5′-end with Fam and at the 3′-end with Tamra):

| | |
|---|---|
| ε forward primer: | |
| ATCCACAGGCACCAAATGGA | (SEQ ID NO:51) |
| ε reverse primer: | |
| GGAAGACGGATGGGCTCTG | (SEQ ID NO:52) |
| ε probe: | |
| ACCCGGCGCTTCAGCCTCCA | (SEQ ID NO:53) |

The probe spans a splice junction and thus will not detect contaminating DNA in the reaction assay. The One-step RT-PCR kit (Applied Biosystems, Foster City, Calif.) was used for the RT-PCR reaction. To normalize the results, the housekeeping gene GAPDH (Applied Biosystems, Foster City, Calif.) was used. The epsilon inhibition ratio R was calculated by dividing the transcript expression values in the presence of Dox and IL-4 (+IL-4/+Dox) by values in the absence of Dox and presence of IL-4(+IL-4/−Dox). This value was considered statistically significant if p<0.05.

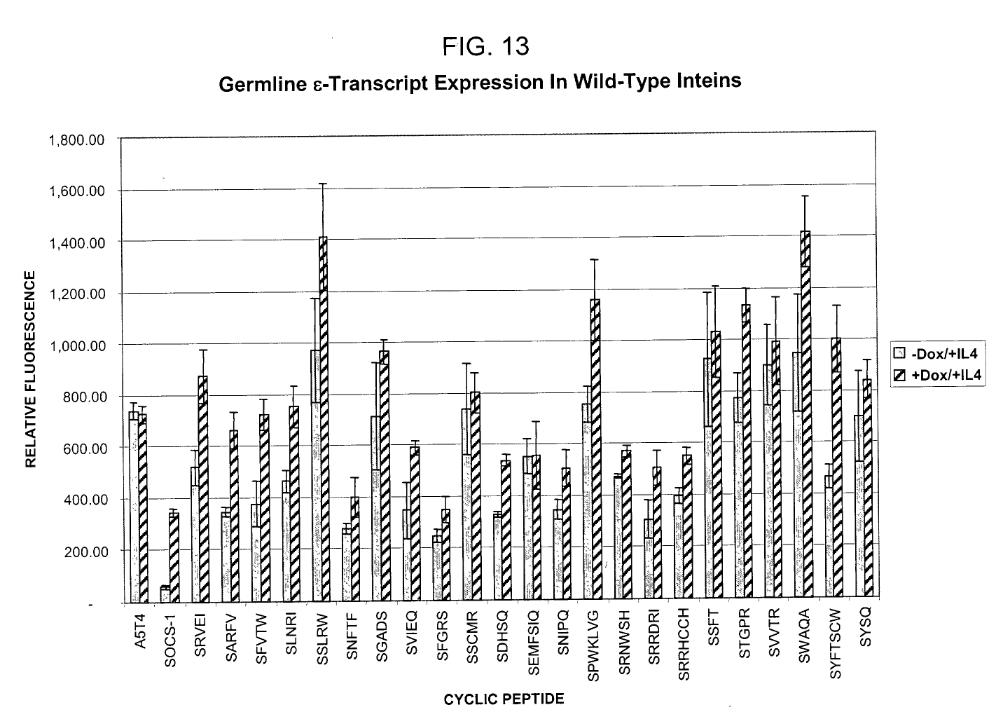

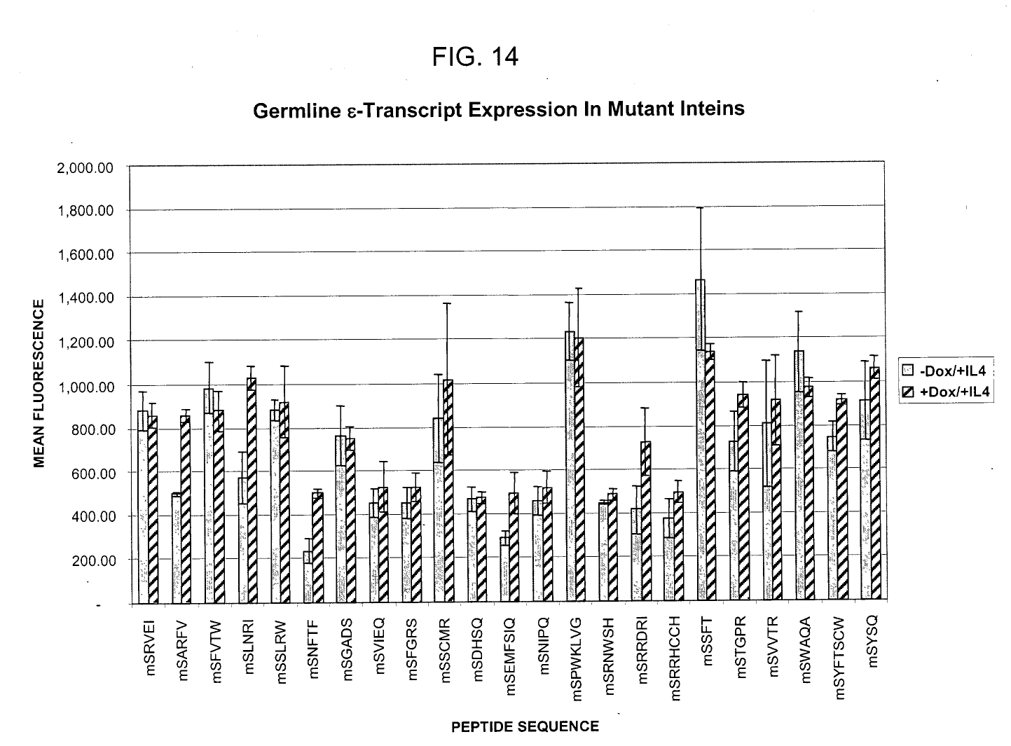

Both the wild type and mutant forms of the inteins were examined. The data for the wild-type clones are presented in FIG. 13; the data for the mutant clones are presented in FIG. 14. In FIG. 13, the known inhibitor SOCS1 was used as a positive control. Uninfected A5T4 cells were used as a negative control. Peptides that were positive for phenotype transfer also showed varying degrees of inhibition of endogenous transcript. Generally, wild-type peptides identified as strong inhibitors by GFP reporter gene inhibition (FIG. 12) also demonstrated the greatest decrease of endogenous transcript (FIG. 13), although none were as potent as the known IL-4 signaling inhibitor SOCS1 (Losman et al., 1999, J. Immunol. 162:3770-3774). The effect of the mutant inteins (FIG. 14) generally mirrored results from GFP reporter gene inhibition (FIG. 12), with the exception of SNFTF, which in the mutant context clearly blocked endogenous ε-transcription but did not strongly block GFP reporter gene inhibition.

7.5 Cyclic Peptides SEQ ID NOS:1-20 Are Selective for the Germline ε Promoter

To demonstrate selectivity for the germline ε promoter, inhibitory cyclic peptides were tested for inhibition of germline ax transcription using quantitative RT-PCR to measure TGF-P induced germline a transcripts in ST486 cells. The assay was similar to that described in the immediately preceding section, except that ST486 cells (ATCC # CRL-1647) engineered to express the LTA-VP16 ires Hygromycin phosphotransferance gene were used and the infected cells were stimulated with TGF-β. The primers and probe used were as follows (the probe was labeled at the 5′-end with Fam and at the 3′-end with Tamra):

| | |
|---|---|
| α forward primer: | |
| CAGCACTGCGGGCCC | (SEQ ID NO:50) |
| α reverse primer: | |
| TCAGCGGGAAGACCTTGG | (SEQ ID NO:51) |
| α probe: | |
| CCAGCAGCCTGACCAGCATCCC | (SEQ ID NO:52) |

As with the epsilon primer/probe set, the alpha probe spans a splice junction and does not detect DNA. None of the cyclic peptides of TABLE 1 inhibited promoter alpha transcription.

7.6 The A5T4 Reporter Cell Line Expresses Cyclic Peptides

To confirm that cyclic peptides are expressed in the A5T4 reporter cell line, a purification method for small cyclic peptides was developed using the synthetic cyclic peptide cyclo(SRGDGWS) spiked into cell lysates of naive A5T4 cells. Briefly, A5T4 BJAB cells (5×10 7) were lysed in 400 uL of PBS (MgCl₂ free) containing 4% Tween 20, 2 mM EGTA, 0.4 mM PMSF and a tablet of protease inhibitors (Roche Biochemicals, Palo Alto, Calif.) by freeze-thaw method. Proteins were precipitated by the addition of TFA to 0.1% v/v. The lysate was centrifuged 30 minutes at 14K (4° C.) to remove precipitated proteins, and the supernatant was passed through a 3 kDa-cutoff filter (Millipore Corporation, Bedford, Mass.) and concentrated by vacuum centrifugation to ca. 50 ul. This solution was injected onto a C18SP 5 cm×1 mm i.d. Vydac (Hesperia, Calif.) reversed phase column eluted on an HP 1100 HPLC (Hewlett Packard, Palo Alto, Calif.) at 100 ul/min. and 1 min. fractions were collected. These were centered around the elution position of the synthetic standard peptide (from American Peptide Co., Sunnyvale, Calif.) when available. One microliter of each fraction was mixed with 1 ul dihydroxybenzoate matrix solution (Agilent, Palo Alto, Calif., diluted 10-fold with 50% v/v acetonitrile-0.1% v/v TFA). One microliter of this mixture was spotted onto a 400 micron well of an Anchor Chip (Bruker, Billerica, Mass.) and the mass spectrum was collected on a Bruker Reflex III time-of-flight mass spectrometer. Masses were assigned after external calibration using angiotensin III and the peptide MRFA; the mass accuracy was 100 ppm. MS/MS spectra were collected on an LCQ ion trap mass spectrometer (ThermoFinnigan, San Jose, Calif.) after injection of the peptide fraction onto a capillary C18 reversed phase column eluted into the mass spectrometer source.

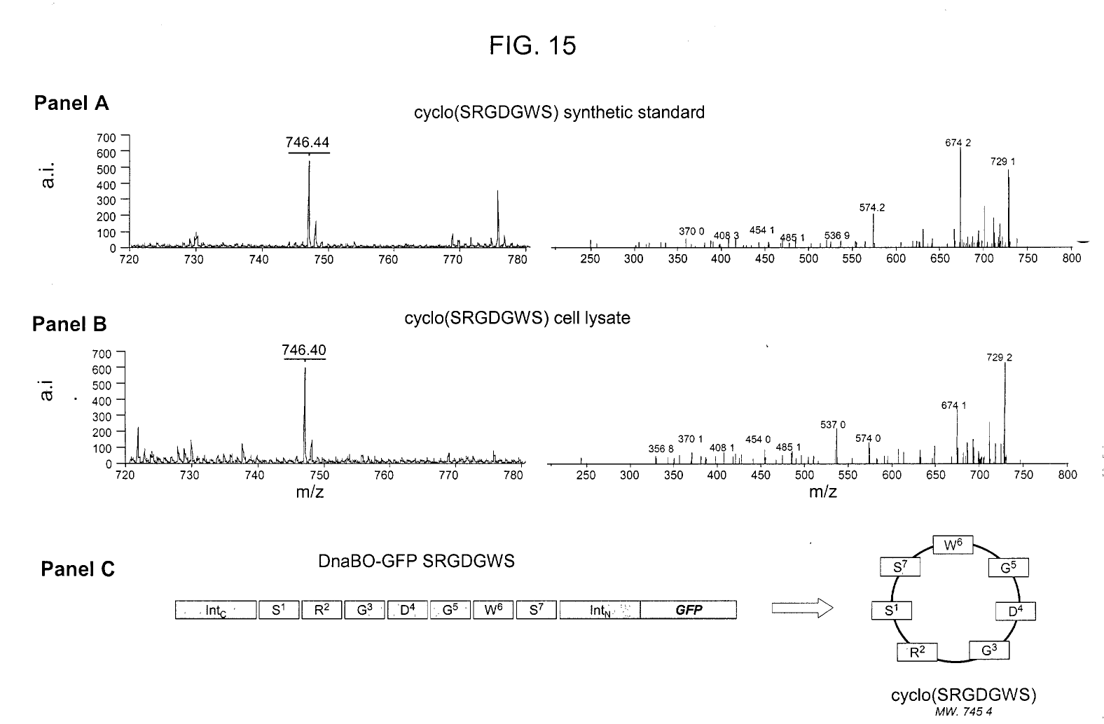

This method was then applied to lysates of A5T4 cells that had been infected with a construct expressing the above cyclic peptide and FACS sorted for GFP fluorescence into a homogeneous population expression the precursor fusion. A peptide with a mass and MS/MS fragmentation pattern nearly identical to the predicted or spiked synthetic peptide was observed by mass spectrometry (see FIG. 15). In FIG. 15, Panel A provides MALDI-TOF data (left side) and MS/MS data (right side) for cell lysates spiked with 100 pmol of the synthetic cyclo(SRGDGWS) synthetic standard. Similar data for cell lysates expressing cyclo(SRGDGWS) via the intein construct illustrated in Panel C are provided in Panel B.

This method was used to examine cell lysates for the presence of selected cyclic peptides using MALDI-TOF mass spectrometry. The data are presented in TABLE 2, below:

TABLE 2

| | Uninfected A5T4 | | |
|---|---|---|---|
| | cells spiked with | A5T4 cells infected | A5T4 cells infected |
| Cyclic | synthetic peptide | with wild type intein | with Mutant intein |
| peptide | (MH+) | (MH+ theor/obs) | (MH+ theor/obs) |
| SARFV | 561.36 | 561.40/561.32 | none observed |
| SRVEI | N/A | 585.34/585.34 | none observed |
| SFVTW | 621.34 | none observed | none observed |
| SLNRI | 584.41 | none observed | none observed |
| SSLRW | 630.43 | 630.43/630.47 | none observed |
| SNFTF | N/A | 597.27/597.26 | none observed |
| SGADS | N/A | none observed | none observed |
| SVIEQ | N/A | none observed | none observed |
| SFGRS | N/A | none observed | none observed |
| SSCMR | N/A | none observed | none observed |

For SRVEI, SARFV, SSLRW and SNFTF, the results were consistent with production of the cyclic peptide from the active inteins. Cyclic SSLRW showed the highest intracellular concentrations and when its peak intensity was compared to that of its synthetic counterpart spiked into uninfected cell lysates, we could estimate its cellular concentrations and when its peak intensity was compared to that of its synthetic counterpart spiked into uninfected cell lysates, we could estimate its cellular concentrations to be ca. 0.5 μM (data not shown). This is similar to levels of GFP-fused peptide library members in A549 and Jurkat cells (Peelle et al., 2001, Chem. Biol. 8:521-534). No cyclic peptide was observed for SFVTW, SLNRI, SGADS, SVIEQ, SFGRS and SSCMR. This could be attributed to the fact that the intracellular concentrations may be below our detection capabilities. Alternatively, these peptides may be undergoing partial splicing and may be active through an intermediate lariat structure (Perler & Adam, 2000, Curr. Opin. Biotechnol. 11:377-383). Taken together, the results demonstrate that the functional screen was able to select for peptide library members capable of producing cyclic peptides that inhibited endogenous i-promoter activity.

While the invention has been described by reference to various specific embodiments, skilled artisans will recognize that numerous modifications may be made thereto without departing from the spirit and the scope of the appended claims.

All references cited throughout the disclosure are incorporated herein by reference in their entireties for all purposes.

What is claimed is:

1. A cyclic compound composed of from 4 to 10 residues comprising a contiguous sequence of residues selected from structures (Ia)-(Ie):
X²⁶˜X²⁵˜X²¹˜X²⁵; (Ia)
X²²˜X²⁴˜X²⁷˜X²⁹; (Ib)
X²¹˜X²²˜X²⁴˜X²⁹; (Ic)
X²²˜X²¹˜X²¹˜X²⁹; (Id)
X²⁸˜Glu˜X²¹˜X^{29;}and (Ie)
pharmaceutically acceptable salt thereof, wherein:
each X²¹ is independently a small residue;
each X²² is independently a basic residue;
each X²⁴ is independently a hydrophobic residue;
each X²⁵ is independently an aromatic residue;
each X²⁶ is independently a polar residue;
each X²⁷ is independently a small or a cysteine-like residue;
each X²⁸ is independently a small or an acidic residue; and
each X²⁹ is independently any residue; and
each “˜” independently represents an amide, a substituted amide, an isostere of an amide or an amide or peptido mimetic linkage.

2. The cyclic compound of claim 1 which is selected from the group consisting of structures (IIa)-(IIe):
cyclo(X²⁶˜X²⁵˜X²¹˜X²⁵˜X³⁰˜X³⁰˜X³⁰) (IIa)
cyclo(X²²˜X²⁴˜X²⁷˜X²⁹˜X³⁰˜X³⁰˜X³⁰) (IIb)
cyclo(X²¹˜X²²˜X²⁴˜X²⁹˜X³⁰˜X³⁰˜X³⁰) (IIc)
cyclo(X²²˜X²¹˜X²¹˜X²⁹˜X³⁰˜X³⁰˜X³⁰) (IId)
cyclo(X²⁸˜Glu˜X²¹˜X²⁹˜X³⁰˜X³⁰˜X³⁰); and (IIe)
pharmaceutically acceptable salts thereof, wherein:
X²¹X²²X²³X²⁴X²⁵X²⁶X²⁷X²⁸X²⁹ and “˜” are as defined for claim 1 and each X³⁰ is independently present or absent, and if present, is any residue.

3. The cyclic compound of claim 2 which is structure (IIa).

4. The cyclic compound of claim 3 in which:
each X²¹ is independently selected from the group consisting of a Ser, a Thr, a Cys and a Asn residue; and/or
each X²⁵ is independently selected from the group consisting of a Trp, a Phe, a Tyr and a His residue.

5. The cyclic compound of claim 3 which has the structure (IIá)
cyclo (X⁴⁰˜X⁴¹˜X⁴²˜X⁴³˜X³⁰X³⁰ X³⁰) (IIá)
wherein:
X³⁰ is as defined in claim 3;
X⁴⁰ is a Ser, a Thr, a Cys or a Asn residue;
X⁴¹ is a Trp, a Phe, a Tyr or a His residue;
X⁴² is a Ser or a Thr residue; and
X⁴³ is a Phe, a Tyr, a His or a Trp residue.

6. The cyclic compound of claim 5 which is composed of 5, 6 or 7 residues.

7. The cyclic compound of claim 5 in which X³⁰˜X³⁰˜X³⁰ is selected from the group consisting of (a Phe residue)˜(a Thr residue)˜(a Ser residue), (a Ser residue)˜(an Arg residue), a Ser residue and a Val residue.

8. The cyclic compound of claim 5 which is selected from the group consisting of cyclo(TWSFV) cyclo(SEQ ID NO:1), cyclo(CWSYFTS) cyclo(SEQ ID NO:5), cyclo(NWSHSR) cyclo(SEQ ID NO:9), cyclo(NFTFS) cyclo(SEQ ID NO:10) and retro and retro-inverso peptides thereof.

9. The cyclic compound of claim 2 which is structure (IIb).

10. The cyclic compound of claim 9 which is composed of 5, 6 or 7 residues.

11. The cyclic compound of claim 9 in which:
X²² is an Arg, a Lys or an Orn residue;
X²⁴ is an aromatic residue or a small aliphatic residue; and/or
X²⁷ is a small hydroxyl-containing or a Cys residue.

12. The cyclic peptide of claim 9 in which X²⁹˜X³⁰˜X³⁰˜X³⁰ is selected from the group consisting of (hydroxyl-containing residue)˜(aliphatic residue), (aliphatic residue)˜(Asn residue), (basic residue)˜(basic residue)˜(acidic residue) and (sulfanyl-containing residue)˜(basic aromatic residue)˜(small hydroxyl-containing residue)˜(basic residue).

13. The cyclic peptide of claim 8 which is selected from the group consisting of cyclo(RWSSL) cyclo(SEQ ID NO:6), cyclo(RISLN) cyclo(SEQ ID No;4), cyclo(RISRRD) cyclo(SEQ ID NO:15) and retro and retro-inverso peptides thereof.

14. The cyclic compound of claim 2 which is structure (IIc).

15. The cyclic compound of claim 14 which is composed of 5 residues.

16. The cyclic compound of claim 14 in which X²⁹˜X³⁰˜X³⁰˜X³⁰ is selected from the group consisting of (aliphatic residue)(acidic residue) and (aliphatic residue)(hydroxyl-containing residue).

17. The cyclic compound of claim 16 in which:
X²¹ is small hydroxyl-containing residue or a small aliphatic residue;
X²² is an Arg, a Lys or an Orn residue; and/or
X²⁴ is a small aliphatic residue or a hydrophobic residue.

18. The cyclic compound of claim 14 which is selected from the group consisting of cyclo(SRVEI) cyclo(SEQ ID NO:2), cyclo(ARFVS) cyclo(SEQ ID NO:3) and retro and retro-inverso peptides thereof.

19. The cyclic compound of claim 2 which is structure (IId).

20. The cyclic compound of claim 19 which is composed of 5 residues.

21. The cyclic compound of claim 20 in which X²⁹˜X³⁰˜X³⁰˜X³⁰ is selected from the group consisting of (aromatic residue)˜(small residue), (small residue)˜(structurally constrained residue), and (small hydroxyl- or sulfanyl-containing residue)˜(Met residue).

22. The cyclic compound of claim 21 in which:
X²² is an Arg residue; and/or
each X²¹ is independently a small hydroxyl-containing residue.

23. The cyclic compound of claim 19 which is selected from the group consisting of cyclo(RSSFG) cyclo(SEQ ID NO:7), cyclo(RSTGP) cyclo(SEQ ID NO:16) and retro and retro-inverso peptides thereof.

24. The cyclic compound of claim 2 which is structure (IIe).

25. The cyclic compound of claim 24 which has the structure (IIe′):
cyclo(X⁵⁰˜Glu˜X⁵¹˜X²⁹X³⁰˜X³⁰˜X³⁰) (IIe′)
wherein:
X²⁹ and X³⁰ are as defined in claim 2;
X⁵⁰ is a Glu, a Ser or an Ala residue; and/or
X⁵¹ is a Ser or an Ala residue.

26. The cyclic compound of claim 24 or 25 which is composed of 5 residues.

27. The cyclic compound of claim 26 in which X²⁹˜X³⁰˜X³⁰˜X³⁰ is selected from the group consisting of aromatic residue, (aliphatic residue)˜(aliphatic residue), (small hydroxyl-containing residue)˜(aromatic residue) and (small acidic residue)˜(aromatic residue).

28. The cyclic compound of claim 24 which is selected from the group consisting of cyclo(EQSVI) cyclo(SEQ ID NO:13), cyclo(SQSY) cyclo(SEQ ID NO:14), cyclo(AQASW) cyclo(SEQ ID NO:19) and retro and retro-inverso peptides thereof.

29. The cyclic compound of claim 1 which is selected from the group consisting of:
cyclo(X¹⁶˜X⁵˜X¹⁸˜X¹⁷˜X¹⁹);
cyclo(X¹⁶˜X¹⁵˜X¹⁸˜X⁴˜X⁸);
cyclo(X¹⁶˜X¹˜X¹⁵˜X⁵˜X¹⁸);
cyclo(X¹⁶˜X¹⁰˜X¹²˜X¹⁵˜X⁸);
cyclo(X¹⁶˜X²⁰˜X⁵˜X¹⁷˜X¹⁶˜X²˜X¹⁹);
cyclo(X¹⁶˜X¹⁶˜X¹⁰˜X¹⁵˜X¹⁹);
cyclo(X¹⁶˜X⁵˜X⁶˜X¹⁵˜X¹⁶);
cyclo(X¹⁶˜X⁴˜X¹¹˜X⁵˜X¹⁶˜X⁸˜X¹⁹);
cyclo(X¹⁶˜X⁴˜X¹¹˜X⁵˜X¹⁶˜X⁷);
cyclo(X¹⁶˜X¹²˜X⁵˜X¹⁷˜X⁵);
cyclo(X¹⁶˜X¹²˜X⁸˜X¹³˜X¹⁴);
cyclo(X¹⁶˜X⁶˜X¹˜X³˜X¹⁶);
cyclo(X¹⁶˜X¹⁸˜X⁸˜X⁴˜X¹⁴);
cyclo(X¹⁶˜X²⁰˜X¹⁶˜X¹⁴);
cyclo(X¹⁶˜X¹⁵˜X¹⁵˜X³˜X¹⁵˜X⁸);
cyclo(X¹⁶˜X¹⁷˜X⁶˜X¹³ ˜X¹⁵);
cyclo(X¹⁶˜X¹⁸˜X¹⁸˜X¹⁷˜X¹⁵);
cyclo(X¹⁶˜X¹³˜X¹⁹˜X⁹˜X¹⁰˜X¹⁸˜X⁶);
cyclo(X¹⁶˜X¹⁶˜X¹˜X¹⁴˜X¹); and
cyclo(X¹⁶˜X³˜X⁷˜X¹⁶˜X¹⁴);
wherein:
X¹ is an Ala residue;
X² is a Cys residue;
X³ is an Asp residue;
X⁴ is a Glu residue;
X⁵ is a Phe residue;
X⁶ is a Gly residue;
X⁷ is a His residue;
X⁸ is an Ile residue;
X⁹ is a Lys residue;
X¹⁰ is a Leu residue;
X¹¹ is a Met residue;
X¹² is an Asn residue;
X¹³ is a Pro residue;
X¹⁴ is a Glu residue;
X¹⁵ is an Arg residue;
X¹⁶ is a Ser residue;
X¹⁷ is a Thr residue;
X¹⁸ is a Val residue;
X¹⁹ is a Trp residue;
X²⁰ is a Tyr residue; and
retro- and retro-inverso analogs thereof.

30. The cyclic compound of claim 1 or claim 29 in which all residues having a chiral α-carbon are in the same configuration about the α-carbon.

31. The cyclic compounds of claim 30 in which all residues having a chiral α-carbon are in the L-configuration about the α-carbon.

32. The cyclic compounds of claim 1, 2 or 30 which are peptides.

33. The cyclic compounds of claim 32 which are composed wholly of gene-encoded amino acids.

34. The cyclic compounds of claim 1, 2 or 30 which are peptide analogs.

35. The cyclic compounds of claim 34 which have enhanced in vivo stability compared to a corresponding cyclic peptide composed wholly of gene-encoded amino acids.

36. The cyclic compounds of claim 1, 2 or 30 which are labeled.

37. The cyclic compounds of claim 1, 2 or 3 which include a moiety that enhances transport across a membrane.

38. The cyclic compounds of claim 1, 2 or 30 which inhibit at least about 25% of IL-4 induced transcription of a germline ε promoter as compared to control cells.

39. A polynucleotide capable of expressing a cyclic peptide comprising a first segment encoding a C-terminal intein domain, a second segment encoding a linear version of cyclic compound according to claim 33 and a third segment encoding an N-terminal intein domain, wherein the first second and third segments are arranged such that the polynucleotide expresses a cyclic peptide.

40. The polynucleotide of claim 39 which further includes a promoter upstream of the encoding segments.

41. The polynucleotide of claim 35 in which the second segment encodes a peptide selected from the group consisting of:

| | |
|---|---|
| S F V T W; | (SEQ ID NO:1) |
| S R V E I; | (SEQ ID NO:2) |
| S A R F V; | (SEQ ID NO:3) |
| S L N R I; | (SEQ ID NO:4) |
| S Y F T S C W; | (SEQ ID NO:5) |
| S S L R W; | (SEQ ID NO:6) |
| S F G R S; | (SEQ ID NO:7) |
| S E M F S I Q; | (SEQ ID NO:8) |
| S R N W S H; | (SEQ ID NO:9) |
| S N F T F; | (SEQ ID NO:10) |
| S N I P Q; | (SEQ ID NO:11) |
| S G A D S; | (SEQ ID NO:12) |
| S V I E Q; | (SEQ ID NO:13) |
| S Y S Q; | (SEQ ID NO:14) |
| S R R D R I; | (SEQ ID NO:15) |
| S T G P R; | (SEQ ID NO:16) |
| S V V T R; | (SEQ ID NO:17) |
| S P W K L V G; | (SEQ ID NO:18) |
| S W A Q A; and | (SEQ ID NO:19) |
| S D H S Q. | (SEQ ID NO:20) |

42. A host cell or progeny thereof comprising a polynucleotide according to claim 39.

43. A pharmaceutical composition comprising a cyclic compound according to claim 1 and a pharmaceutically acceptable carrier, excipient or diluent.

44. A method of inhibiting IgE production in a B-cell, or a process associated therewith, comprising administering to the cell a compound according to claim 1.

45. The method of claim 44 in which the process inhibited is IL-4 induced germline ε transcription.

46. The method of claim 44 in which the process inhibited is IL-4 induced isotype switching to produce IgE.

47. The method of claim 44 in which the cyclic compound is administered to the cell via the polynucleotide of claim 39.

48. A method of treating or preventing a disease associated with, caused by or mediated by IgE production and/or accumulation, or symptoms associated therewith, comprising administering to an animal suffering from such a disease an amount of a cyclic compound according to claim 1 effective to treat or prevent the disease or its associated symptoms.

49. The method of claim 48 in which the disease is selected from the group consisting of an anaphylactic or hypersensitivity reaction, atopic dermatitis, atopic eczema, atopic asthma, allergic rhinitis, allergic conjunctivitis, systemic mastocytosis, hyper IgE syndrome, IgE gammopathies and B-cell lymphoma.

50. The method of claim 48 in which the cyclic compound administered is a compound according to claim 32.

51. The method of claim 48 in which the cyclic compound is administered via administration of a polynucleotide capable of expressing the cyclic compound in the animal.

52. The method of claim 48 in which the animal is a human.
